(19) United States
(12) Patent Application Publication
(10) Pub. No.: US 2025/0311721 A1
(43) Pub. Date: Oct. 9, 2025

(54) UREA COMPLEXES OF ACTIVE INGREDIENTS

(71) Applicant: BAYER CROPSCIENCE LP, St. Louis, MO (US)

(72) Inventors: David Morgenstern, St. Louis, MO (US); Gary Kenneth Windler, Jr., St. Louis, MO (US)

(21) Appl. No.: 18/865,154

(22) PCT Filed: May 11, 2023
(86) PCT No.: PCT/US2023/021883

§ 371 Date: Nov. 12, 2024

Related U.S. Application Data
(60) Provisional application No. 63/341,649, filed on May 13, 2022.

(51) Int. Cl. A01N 33/00; A01N 31/02; A01N 39/00; A01N 43/16; A01N 57/12; A01P 3/00; A01P 5/00; A01P 13/00; A01P 17/00

(52) U.S. Cl. CPC A01N 33/00; A01N 31/02; A01N 39/00; A01N 43/16; A01N 57/12; A01P 3/00; A01P 5/00; A01P 13/00; A01P 17/00

(57) ABSTRACT

The present invention relates to the technical field of delivery systems for agrochemical active ingredients or pharmaceutical active ingredients. The present invention primarily relates to certain compositions in solid form comprising as constituent (a) at least one agrochemical or pharmaceutical active ingredients, as constituent (b) urea, and as constituent (c) at least one non-ionic surfactant. The present invention also relates to products obtainable or obtained by the processes defined in the context of the present invention. The present invention further relates to formulations comprising these compositions or products and application mixtures obtainable or obtained by dilution of these compositions or products with water or aqueous surfactant solutions. The present invention further relates to processes for preparing these compositions, products, formulations and application mixtures as well as to uses thereof and methods of using these compositions, products, formulations and application mixtures.

### CROSS-REFERENCE TO RELATED APPLICATIONS

This application claims the benefit of, and priority to, U.S. Provisional Application No. 63/341,649, filed May 13, 2022, the entire contents of all of which are hereby incorporated by reference as if fully set forth herein.

### FIELD OF THE INVENTION

The present invention relates to the technical field of delivery systems for agrochemical active ingredients or pharmaceutical active ingredients. The present invention primarily relates to certain compositions in solid form comprising as constituent (a) at least one agrochemical or pharmaceutical active ingredients, as constituent (b) urea, and as constituent (c) at least one non-ionic surfactant. The present invention also relates to products obtainable or obtained by the processes defined in the context of the present invention. The present invention further relates to formulations comprising these compositions or products and application mixtures obtainable or obtained by dilution of these compositions or products with water or aqueous surfactant solutions. The present invention further relates to processes for preparing these compositions, products, formulations and application mixtures as well as to uses thereof and methods of using these compositions, products, formulations and application mixtures.

### BACKGROUND OF THE INVENTION

Delivery of poorly soluble active ingredients (actives) to the target organism is a major challenge in both agriculture and pharmacology. In virtually all cases, efficacy requires that the active molecule reach the circulatory system of a plant (for herbicides and systemic fungicides and insecticides) or a human or veterinary patient. In pharmaceutical science, this is typically achieved via Amorphous Solid Dispersions, as described for example in Int. J. Pharm. 2020, 586, 11950. The Active Pharmaceutical Ingredient (API) is impregnated into a polymer matrix using a solvent which is subsequently removed. The API diffuses out of the polymer and dissolves in the stomach or small intestine of the patient, a process enabled by the fact that the active is in a non-crystalline form in the polymer matrix. This accelerates dissolution and can achieve super-saturated concentrations of the active. Similar formulation strategies, such as impregnation on silica, are also known in pharmacology.

Although many active, low-solubility active ingredients exist in the field of agriculture, matrix encapsulation strategies of the type used for pharmaceutical actives are ineffective in practice. Agricultural pesticides are almost always sprayed on target plants or soil through a water spray. Unlike pharmaceutical delivery in which the matrix particle may be in the gastrointestinal tract for hours, agricultural formulations are added to the spray mixture immediately before application, often a period of just a few minutes. After spraying and the rapid drying of the spray solution, the active is in contact only with a thin layer of adventitious moisture on the leaf or soil surface. This inhibits transport of the active to the interior of the plant and distribution in the soil.

Several strategies are known in the art for enhancing the uptake of pesticides with low solubility, particularly low water solubility. In some cases, it is possible to dissolve a sufficient amount of active in a suitable solvent that can carry the active into the leaf, for example as a formulation type known as an emulsifiable concentrate. Most often, however, the active is milled to the size of a few microns, suspended in water, and added to the spray tank. Delivery of active from this formulation type, known as a suspension concentrate, is often enhanced by addition of a separate solvent to the spray mixture, typically a crop oil concentrate or methyl ester of soy or rapeseed oil.

While these methods are widely practiced in commercial agriculture, they have significant limitations. Crop oil concentrates and other additives add cost and complexity to the application process and provide inconsistent results especially on soils. Oils adjuvants are not helpful for seed treatment. Moreover, for particularly insoluble actives, solubilization by the additives are insufficient. Thus, there is a need in agriculture for a method to rapidly release low-solubility agricultural actives, typically pesticides, in the spray mixture as fine, low-crystallinity particles which exhibit enhanced solubility and delivery of the active to the plant, soil, fungal, or other target.

Today, the majority of new active ingredients, in particular of active agrochemical ingredients or of pharmaceutical ingredients, shows the properties of poor solubility and subsequently reduced bioavailability. In the field of Active Pharmaceutical Ingredients (APIs), one approach to overcome these issues is embedding the amorphous API into water-soluble polymers forming an Amorphous Solid Dispersion (ASD) since these can increase the oral bioavailability of poorly soluble drugs. Once these systems get into contact with gastrointestinal media, dissolution will occur to a supersaturated state, which is more or less stabilized by the polymer. This so-called “spring and parachute” approach has been shown to significantly enhance the bioavailability of poorly water-soluble APIs. One major challenge in administration of these APIs is the high inter-individual variability of drug performance. Another inherent issue of amorphous solid dispersions is the instability of the solid state which results in a tendency for recrystallisation of the drug and/or excipients during storage. This may be accompanied by a break-down of dissolution and bioavailability.

IN 369969 relates to urea complexes of the insecticides chlorpyriphos, malathion, bifenthrin and cypermethrin for improving safe handling and other characteristics.

U.S. Pat. No. 4,065,289 discloses herbicidal compositions containing a plant fertilizer.

U.S. Pat. No. 5,714,157 teaches certain water-dispersible granular agricultural compositions comprising an active ingredient, a base, urea, an urea modifier, and optionally further additives that are formed by extrusion.

U.S. Pat. No. 5,474,971 concerns certain rapidly disintegrating granular compositions made by extruding a dry premix through a die or screen at elevated temperature comprising an active ingredient, a water-soluble diluent and at least two further additives.

WO 2014/093522 discloses to (a method for producing) extruded pesticide granular compositions suitable for preparing a near stable micro-emulsion, said granular compositions comprising urea, a non-ionic surfactant, a pesticide active ingredient, and water.

US 2016/0050913 relates a method of manufacturing certain agricultural and horticultural granule formulations via molding a powdered composition into granules with a lateral extrusion granulator.

J. Pharm. Sci. 1966, (55), 581-583 investigates the dissolution rates and gastrointestinal absorption of chloramphenicol-urea samples via solid solutions and eutectic mixtures.

IN 182620 concerns a process for the preparation of urea complexes of vitamin E and its esters.

International Journal of Pharmaceutics 1997, 156, 175-180 had the objective to improve the dissolution rate of the sparingly water-soluble drug ofloxacin by solid dispersion systems with urea or mannitol.

J. Pharm. Sci., 2008, Vol. 97, No. 3, 1191-1201 reports on the adduction of amiloride hydrochloride in urea.

J. Pharmacy Pharmacology, 2007, 59, 1501-1507 discloses urea inclusion compounds of enalapril maleate for the improvement of pharmaceutical characteristics.

J. Incl. Phenom. Macrocycl. Chem. 2008, 60, 203-209 studied urea co-inclusion compounds of glipizide for the improvement of dissolution profile.

J. Pharm, Innov., 2008, 3, 249-57 investigated the use of hexagonal urea as a means for the reduction in moisture sensitivity/uptake of moisture sensitive drugs through adduction in urea using nicorandil as model drug.

Powder Technology 2014, 257, 168-174 evaluated clarithromycin-urea solid dispersions prepared by solvent evaporation, electrospraying and freeze drying methods.

J. Incl. Phenom. Macrocycl. Chem. 2015, 81, 105-120 reports on studies on urea co-inclusion complexes of simvastatin for improvement of pharmaceutical characteristics.

Drug Development and Industrial Pharmacy 2015, 41 (9), 1401-1415 provides a review on the classification of solid dispersions: correlation to (i) stability and solubility (ii) preparation and characterization techniques.

Drug Delivery 2020, Vol. 27, No. 1, 110-127 reviews the mechanism of increased bioavailability through amorphous solid dispersions.

Mol. Pharmaceutics 2021, 18, 1905-1919 reports on the characterization of Amorphous Solid Dispersion (ASDs).

WO 95/08987 pertains to a process for preparation of solid dispersions and deposits as well as of solid forms with dihydropridine type calcium antagonists, such as for example nimodipine.

WO 2021/156172 relates to pharmaceutical compositions containing regorafenib and a stabilizing agent.

### BRIEF DESCRIPTION OF THE INVENTION

It was found that compositions in solid form based on urea and comprising active ingredients with certain structural and physical properties and non-ionic surfactants in certain amounts and ratios, allow quick dissolution in water or aqueous diluents, resulting in enhanced or improved bioavailability of the active ingredient.

Briefly, certain aspects of the present invention are directed to certain solid compositions comprising (a) a total amount of at least about 5% by weight of one or more pesticidal or pharmaceutical active ingredients having a melting point of at least 55° C. and a solubility of 50 g/L or less in deionized water, said constituent (a) not being present in the form of a salt with an inorganic counter-ion, (b) a total amount of at least about 50% by weight of urea, and (c) a total amount of at least about 1% by weight of one or more non-ionic surfactants, wherein the ratio by weight of the total amount of constituent (c) to the total amount of constituent (a) is about 0.8 or lower.

In certain embodiments, the solid compositions according to the present invention are in the form of an inclusion complex or a solid solution. In other embodiments, the compositions in solid form according to the present invention, upon dilution with water, yield nanoparticles of the active ingredient or yield the active ingredient in liquid form. In still further embodiments, the solid compositions according to the present invention form, upon adding to an aqueous solution of an emulsifying surfactant, yield the active ingredient in emulsified form.

Other aspects of the present invention are directed to products obtainable or obtained from these solid compositions by methods like (hot melt) extrusion, melting and cooling, spray drying, spray chilling, prilling, spheronization, or combinations thereof.

In further aspects, the present invention relates to a process for preparing a composition according to the present invention, and to application mixtures obtained or obtained from compositions or products according to the present invention.

Further aspects of the present invention are directed to methods for controlling undesired vegetation, plant pests, (phytopathogenic) fungi or (phytopathogenic) nematodes and to the corresponding uses.

Further aspects of the present invention are directed to composition, product or application mixture defined in the context of the present invention for use as a medicament, for use in the treatment of an animal or human body, and to corresponding methods and uses.

### DETAILED DESCRIPTION OF THE INVENTION

Generally, the present invention relates to compositions which are solid form at 25° C. and 1013 mbar comprising:
(a) a total amount of at least about 5% by weight of one or more pesticidal or pharmaceutical active ingredients having a melting point of at least 55° C. at 1013 mbar and a solubility of 50 g/L or less in deionized water having a pH of about 7 at 25° C. and 1013 mbar, constituent (a) not being present as a salt with an inorganic counter-ion, (i.e. constituent (a) is not present as salt with an inorganic counter-ion such as monovalent metal ions, divalent metal ions, trivalent metal ions or an ammonium counter-ion),
(b) a total amount of at least about 50% by weight of urea,
(c) a total amount of at least about 2% by weight of one or more non-ionic surfactants, wherein the ratio by weight of the total amount of constituent (c) to the total amount of constituent (a) is about 0.8 or lower, and
wherein the amounts indicated in each case are based on the total weight of the composition.

In the context of the present invention, the compositions or products of the present invention sometimes are also referred to as complexes.

In the context of the present invention many embodiments of compositions or products of the present invention are defined by the amounts of the constituents comprised therein. These amounts are typically indicated in ranges in percent by weight (wt. %) based on the total weight of the composition. It is understood by the skilled artisan that the sum of these amounts does not (and by definition cannot) exceed 100%.

The compositions of the present invention primarily comprise one or more pesticidal or pharmaceutical active ingredients having the above-mentioned properties, typically agricultural pesticides, in urea along with non-ionic surfactants, and optionally further constituents such as dispersants that aid is the wetting and dissolution of the urea particle matrix and the dispersion of the active ingredient. Importantly, urea is extremely soluble in water (about 100 g per 100 ml water) and dissolves in seconds. The active is released in this process rapidly, for example forming small, disordered particles that are more bioavailable than crystalline solid milled to form a suspension concentrate. The use of certain polymeric dispersants promotes the formation of particularly small particles of some actives as described in more detail the Examples hereinafter. In particularly favorable cases, the active does not crystallize very fast and forms an oil that is dispersed in water.

In practice, the complexes of the present invention can be prepared from molten or near-molten urea in which the one or more actives of constituent (a) are dissolved, then cooled quickly enough, typically in under a minute, to prevent or minimize phase separation as the urea solidifies. In some cases, fine amorphous particles of the one or more actives of constituent (a) present in the urea matrix prior to hydrolysis were observed, which is also satisfactory since these particles, when released, also provide enhanced bioavailability. This process is compatible with well-known and commercially practiced methods for production of agricultural formulations at scale including (hot melt) extrusion, spray drying, and prilling. In addition, the process is suitable for those pharmaceutical actives where the relatively high loading and fast release of the active compared to amorphous solid dispersions is beneficial.

The methods and compositions known from the prior art pesticides can be co-extruded with urea forming a mechanical mixture (“intimate dispersion”) of pesticides with melting points below that of urea. A solid solution of the pesticide in these cases is not formed. In other processes from the prior art urea and active ingredient particles are being mixed and thermally fused rather than forming a single phase.

The distinction between the present invention and the prior art, in which urea typically is used as a binder or “carrier” for active particles, is the clear evidence of molecule-scale interaction between urea and the pesticide, which can be described as “solvation.” In the prior art there is no suggestion of intermingling of urea and the active at a molecular level: the urea sometimes acts only as a coating and carrier for the active particulates, not as a “solvent” for the active.

By contrast, the complexes of the present invention typically are prepared from a molten or near-molten mixture of the active ingredient and urea, although it is sometimes advantageous to add a small amount of water to reduce the melting point of urea and thus the processing temperature. As discussed below, one or more further constituents, such as dispersants, wetting agents or other surfactants, are often a constituent of the melt to provide wetting when the complex is added to water and for other purposes. The one or more actives of constituent (a) must be an organic compound not in inorganic salt form, typically with a molecular weight less than 800 Dalton, and a melting point 55° C. or above. The one or more actives of constituent (a) must have sufficient thermal stability for such processing which generally means it must not undergo significant degradation at temperatures in the range of about 75° C. to 110° C. for a period of up to 30 minutes.

The present invention can also be considered an improved version of an approach used both in the agricultural and pharmaceutical field known as “matrix encapsulation.” The active ingredient is dissolved in a water-soluble matrix, typically a polymer, which dissolves in the spray tank (for agricultural sprays) or the stomach (for pharmaceuticals) releasing the active in molecular form. It now has been found that a surprisingly wide range of agricultural pesticides can be incorporated into a urea matrix, for example by forming a solution in molten solution and rapid cooling although other methods have been demonstrated.

The compositions or products of the present invention are different in that these predominantly are a solution of the active in urea, formed in a molten, semi-molten or near-molten (softened) state. Thus, the compositions or products of the present invention can be prepared from the melt by casting on a surface or melt extrusion, both described below, or for example by prilling and spray drying or by hot melt extrusion. In accordance with the present invention, the particles of the active ingredient form primarily when the urea is dissolved in water or an aqueous surfactant solution (such as for example when producing an application mixture) instead of being present as a mechanical mixture with urea. In this way, the actives are obtained in much smaller particles sizes and with improved bioavailability compared to the prior art.

The objective of the present invention was not only to achieve fast release of the active from the urea complex and to achieve high or improved bioavailability of the active, but also to allow higher efficacy of the active and in particular high loadings of the active in the composition or complex. Such higher loadings are achievable in the present invention the urea matrix because the dissolution process is conducted at elevated temperature. Loading of a conventional emulsifiable concentrate (EC) formulation is limited by the room temperature (about 25° C.) solubility of the active.

The urea complexes (compositions or products) of the present invention comprising a pesticidal active ingredient are superior to prior approaches to enhancing the activity of agrochemical active ingredients having low-solubility in water because urea is much less expensive than the polymers normally used for matrix encapsulation, enables reasonable high loadings of the compositions according to the present invention (typically in the range of from about 10% to about 30% by weight, based in the total weight of the composition or product), and has value both as a plant nutrient and as an adjuvant to improve foliar active ingredient uptake. In addition, when formulated, the urea complexes in powder form exhibit superb dissolution and dispersion properties.

Urea complexes of herbicides such as atrazine, tembotrione, and mesotrione have been prepared. For example, atrazine complexes showed improved control of the weed velvetleaf in the greenhouse. Urea matrix encapsulation according to the present invention has been demonstrated to stabilize mesotrione and tembotrione against decomposition. Also, complexes of fungicides and nematicides have been prepared.

The compositions of the present invention typically comprise
constituent (a) in a total amount of 5% or more by weight, of 6% or more by weight, of 7% or more by weight, typically of 8% or more by weight, more typically of 10% or more by weight, typically in the range of from 5% to 35%, from 6% to 33%, from 7% to 32%, from 8% to 31%, or from 10% to 30%,
and/or
constituent (b) in a total amount of 50% or more by weight, of 60% or more by weight, typically in the range of from 50 to 90% by weight, from 60% to 85% by weight, often in the range of from 65% to 80% by weight,
and/or
constituent (c) in a total amount of 2% or more by weight, 3% or more by weight, 4% or more by weight, of 5% or more by weight, of 8% or more by weight, often of 10% or more by weight,
   wherein the amounts indicated in each case are based on the total weight of the composition.

The compositions of the present invention typically comprise
constituent (a) in a total amount in the range of from 5% to 35%, in the range of from 6% to 33%, or in the range of from 7% to 32%,
and/or
constituent (b) in a total amount in the range of from 50 to 90% by weight, in the range of from 60% to 85% by weight, in the range of from 65% to 80% by weight,
and/or
constituent (c) in a total amount 4% or more by weight, of 5% or more by weight, of 8% or more by weight,
   wherein the amounts indicated in each case are based on the total weight of the composition.

The compositions of the present invention typically comprise
constituent (a) in a total amount in the range of from 7% to 32%, in the range of from 8% to 31%, or in the range of from 10% to 30%,
constituent (b) in a total amount in the range of from 50 to 90% by weight, of from 60% to 85% by weight, or from 65% to 80% by weight, and
constituent (c) in a total amount 5% or more by weight, of 8% or more by weight, or of 10% or more by weight,
   wherein the amounts indicated in each case are based on the total weight of the composition.

The melting point of constituent (a) of compositions according to the present invention typically is in the range of from about 55° C. to about 350° C. at 1013 mbar.

The melting point of constituent (a) of compositions according to the present invention more typically is in the range of from about 60° C. to about 300° C. at 1013 mbar.

The one or more active ingredients of constituent (a) of compositions according to the present invention typically have a molecular weight of less than about 800 Dalton.

The one or more active ingredients of constituent (a) of compositions according to the present invention typically have a molecular weight in the range of from about 200 Dalton to about 800 Dalton.

The one or more active ingredients of constituent (a) of compositions according to the present invention typically have a molecular weight in the range of from about 200 Dalton to about 700 Dalton.

The one or more active ingredients of constituent (a) of compositions according to the present invention more typically have a molecular weight in the range of from about 210 Dalton to about 600 Dalton.

The one or more active ingredients of constituent (a) of compositions according to the present invention more typically have a molecular weight in the range of from about 210 Dalton to about 500 Dalton.

In some case, the one or more active ingredients of constituent (a) of compositions according to the present invention more typically have a molecular weight in the range of from about 300 Dalton to about 600 Dalton.

In some case, the one or more active ingredients of constituent (a) of compositions according to the present invention more typically have a molecular weight in the range of from about 320 Dalton to about 500 Dalton.

The one or more active ingredients of constituent (a) of compositions according to the present invention typically have a solubility of 20 g/L or less in deionized water having a pH of about 7 at 25° C. and 1013 mbar.

The one or more active ingredients of constituent (a) of compositions according to the present invention typically have a solubility of 10 g/L or less in deionized water having a pH of about 7 at 25° C. and 1013 mbar.

The one or more active ingredients of constituent (a) of compositions according to the present invention more typically have a solubility of 5 g/L or less in deionized water having a pH of about 7 at 25° C. and 1013 mbar.

The one or more active ingredients of constituent (a) of compositions according to the present invention even more typically have a solubility of 2 g/L or less in deionized water having a pH of about 7 at 25° C. and 1013 mbar.

The one or more active ingredients of constituent (a) of compositions according to the present invention even more typically have a solubility of 1 g/L or less in deionized water having a pH of about 7 at 25° C. and 1013 mbar.

The one or more active ingredients of constituent (a) of compositions according to the present invention typically have a solubility in acetone of at least about 10 g/L at 25° C. and 1013 mbar.

The one or more active ingredients of constituent (a) of compositions according to the present invention typically have a solubility in acetone of at least about 15 g/L at 25° C. and 1013 mbar.

The one or more active ingredients of constituent (a) of compositions according to the present invention more typically have a solubility in acetone of at least about 25 g/L at 25° C. and 1013 mbar.

The one or more active ingredients of constituent (a) of compositions according to the present invention more typically have a solubility in acetone of at least about 40 g/L at 25° C. and 1013 mbar.

In certain compositions according to the present invention, the ratio by weight of the total amount of constituent (c) to the total amount of constituent (a) typically is in the range of from about 0.1 to about 0.65, based on the total weight of the composition.

In certain compositions according to the present invention, the ratio by weight of the total amount of constituent (c) to the total amount of constituent (a) more typically is in the range of from about 0.2 to about 0.55, based on the total weight of the composition.

In certain compositions according to the present invention, the ratio by weight of the total amount of constituent (c) to the total amount of constituent (a) even more typically is in the range of from about 0.25 to about 0.50, based on the total weight of the composition.

In certain compositions according to the present invention, the total amount of water in the composition typically is less than about 15% by weight, based on the total weight of the composition.

In certain compositions according to the present invention, the total amount of water in the composition more typically is less than about 10% by weight, based on the total weight of the composition.

In certain compositions according to the present invention, the total amount of water in the composition even more typically is less than about 5% by weight, based on the total weight of the composition.

Compositions according to the present invention may contain water, and if present, typically in a total amount in the range of from about 0.1% to about 5% by weight, based on the total weight of the composition.

In certain compositions according to the present invention, the total amount of constituent (c) in the composition typically is at least about 4% by weight, based on the total weight of the composition.

In certain compositions according to the present invention, the total amount of constituent (c) in the composition typically is at least about 5% by weight, based on the total weight of the composition.

In certain compositions according to the present invention, the total amount of constituent (c) in the composition more typically is at least about 6% by weight, based on the total weight of the composition.

In certain compositions according to the present invention, the total amount of constituent (c) in the composition more typically is at least about 7% by weight, based on the total weight of the composition.

In certain compositions according to the present invention, the total amount of constituent (c) in the composition even more typically is at least about 8% by weight, based on the total weight of the composition.

In certain compositions according to the present invention, the total amount of constituent (c) in the composition even more typically is at least about 10% by weight, based on the total weight of the composition.

A precise description of the microstructure of the compositions or products of the present invention is not easy due to the somewhat inconsistent nomenclature used in this technical field. As discussed in the review Chem. Commun., 2014, 50, 904-923, compositions similar to those of the present invention are variously described as “eutectics,” ‘cocrystals,” or “solid solutions”. In case of urea, the terms “inclusion compounds” and clathrate” sometimes are also used.

Eutectics are a long known class of multi-component solids with important and useful applications in daily life. In comparison to other multi-component crystalline solids, such as salts, solid solutions, molecular complexes and cocrystals, eutectics are less studied in terms of molecular structure organization and bonding interactions. Classically, a eutectic is defined based on its low melting point compared to the individual components. The X-ray crystal structure of a cocrystal is different from that of the individual components whereas the unit cell of a solid solution is similar to that of one of the components. Eutectics are closer to the latter species in that their crystalline arrangement is similar to the parent components but they are different with respect to the structural integrity. A solid solution possesses structural homogeneity throughout the structure (single phase), but a eutectic is a heterogeneous ensemble of individual components whose crystal structures are like discontinuous solid solutions (phase separated). Thus, a eutectic may be better defined as a conglomerate of solid solutions. A structural analysis of cocrystals, solid solutions and eutectics has led to an understanding that materials with strong adhesive (hetero) interactions between the unlike components will lead to cocrystals whereas those having stronger cohesive (homo/self) interactions will more often give rise to solid solutions (for similar structures of components) and eutectics (for different structures of components).

The powder X-ray diffraction data obtained so far suggests that the compositions of the present invention retain the general structure of urea and are best described as solid solutions.

Thus, without being bound to one or another literature definition of a “solid solution” or “inclusion complex,” the compositions of the present invention are readily recognized functionally and distinguished from the prior art by their single-phase appearance under a microscope, reduced melting point relative to urea, and most importantly by the rapid release of actives from the urea matrix as liquids or as nanoparticle solids with improved biological activity upon hydrolysis. Seemingly similar materials in the prior art do not share these properties because the urea acts there merely as a binder or carrier for comparatively large active ingredient particulates.

As further described in the Examples, the properties and hydrolysis behavior of the urea compositions and products of the present invention differ significantly from the compositions of the prior art in which urea acts primarily as a binder.

In particular
The release of certain water-insoluble actives from urea as liquids when the compositions or products of the present invention are dissolved in water, even though the actives are room temperature solids. This behavior has not been previously described, and shows that the actives were present as dispersed molecules and not particulates in the urea matrix, since particulates would be released as solid. As described in the Examples hereinafter, this behavior has been observed for urea complexes of the present invention with pesticidal active such as fluopyram, tetraniliprole, and tembotrione;
When examined under a microscope, the single-phase appearance of the compositions and products of the present invention was observed as well as the absence of inclusions. Discrete active particulates larger than about 5 microns can be excluded with confidence in compositions and products of the present invention; and
As evidenced by the substantial absence of discrete particles of the one or more active ingredients when viewed under a microscope when viewed under an optical microscope with sufficient resolution to identify inclusions with diameters of 3 microns or greater. Typically, this requires a magnification of twentyfold (20×).

In the context of the present invention, the term “substantial” or “substantially” refers to 90%, typically 95%, based on the total weight of the composition or product.

The eutectic behavior of compositions and products of the present invention comprising actives such as fluopyram allow extrusion at temperatures around 90° C., well below the melting point of urea or the active alone.

In certain embodiments, the depression of the melting point of a composition or product according to the present invention is below that of pure urea by at least 2° C. as measured by Differential Scanning Calorimetry (DSC). This technique is known in the art and exemplified in Example A9 hereinafter.

In many cases, the compositions and products according to the present invention are a substantially uniform (i.e. wherein constituents (a) and (b), and at least a substantial amount or all of constituent (c), are present in the same phase). Typically, the compositions and products according to the present invention substantially are single-phase compositions or single-phase products, sometimes also referred to in the art as solid dispersion or solid solution. The microstructure depends to a significant extent on the properties of constituent (a).

In this context it has been observed in our analyses that the composition according to the present invention can be in the form of a solid solution in admixture with a portion of solid, non-complexed constituent (a).

In a particularly preferred embodiment, the non-complexed portion comprises an anionic dispersant or a combination of anionic and certain nonionic dispersants. Since ionic compounds such as ionic dispersants are not soluble in urea, these constituents form a separate phase. Preferably the dispersants in this phase are readily water-soluble. The presence of the second phase, comprising anionic dispersant, accelerates the dispersion of the formulation when added to water. It is believed that this phase sub-divides the urea solid solution phase into smaller domains, accelerating their dissolution.

Examples of preferred anionic constituents of the non-complexed phase include alkylated diphenylether disulfonates marketed as sodium salt solutions by Dow (Dowfax®) and Pilot Chemical (Calfax®), mono- and diester-sulfosuccinates such as docusate sodium or Aerosol® surfactants from Solvay, lignosulfonates, alkyl naphthalene sulfonates, and copolymers of maleic acid and alkenes such as Sokalan® CP-9 from BASF.

Examples of polymeric or highly branched nonionic surfactants suitable (also) for the non-complexed phase include tristyrylphenol ethoxylates, castor oil ethoxylates, block copolymers of ethylene oxide and propylene oxide such as the Pluronic® series from BASF, and poly-glycidyl ether polyethylene glycol block copolymer Break-thru® DA 675 from Evonik.

In further embodiments the composition or product according to the present invention is an inclusion complex.

In still further embodiments the composition or product according to the present invention is a solid solution.

In even further embodiments, the composition or product according to the present invention forms nanoparticles of the one or more active ingredients of constituent (a) having a particle size of 100 nm or smaller in diameter, as determined by dynamic light scattering, upon adding the composition or product to water having a pH of about 7 at 25° C. and 1013 mbar in an amount of at least about 5 times the weight of the composition or product, such as about 10 times the weight of the composition or product.

In other embodiments, the one or more active ingredients of constituent (a) of the composition or product according to the present invention are substantially present in the form of a liquid upon adding the composition or product to water having a pH of about 7 at 25° C. and 1013 mbar in an amount of at least about 5 times the weight of the composition or product, such as about 10 times the weight of the composition or product.

In certain other embodiments, the one or more active ingredients of constituent (a) of the composition or product according to the present invention are substantially present in the form of a liquid upon adding the composition or product to a 1% by weight solution of one or more emulsifying surfactants, for example and typically tristyrylphenol ethoxylates, in water at 25° C. and 1013 mbar in an amount of at least about 5 times the weight of the composition or product, such as about 10 times the weight of the composition or product.

In certain other embodiments, the one or more active ingredients of constituent (a) of the composition or product according to the present invention are substantially present in the form of a liquid upon adding the composition or product to water having a pH of about 7 at 25° C. and 1013 mbar or to a 1% by weight solution in water of an emulsifying surfactant (for example and typically tristyrylphenol ethoxylates), at 25° C. and 1013 mbar in an amount of at least about 5 times the weight of the composition or product, such as about 10 times the weight of the composition or product, wherein the active ingredient(s) of constituent (a) are substantially present in the form of an (emulsified) liquid for at least 30 minutes.

It should be noted that oleic acid, which was used in some urea-based compositions described in the prior art, is not a non-ionic surfactant.

Typically, in compositions or products according to the present invention at least one non-ionic surfactant of constituent (c) has a hydrophilic-lipophilic-balance (HLB) value of about 3 or higher.

Typically, in compositions or products according to the present invention at least one non-ionic surfactant of constituent (c) has a hydrophilic-lipophilic-balance (HLB) value of about 4 or higher.

Typically, in compositions or products according to the present invention at least one non-ionic surfactant of constituent (c) has a hydrophilic-lipophilic-balance (HLB) value of about 5 or higher.

Typically, in compositions or products according to the present invention at least one non-ionic surfactant of constituent (c) has a hydrophilic-lipophilic-balance (HLB) value of about 6 or higher.

More typically, in compositions or products according to the present invention constituent (c) has a hydrophilic-lipophilic-balance (HLB) value of about 3 or higher.

More typically, in compositions or products according to the present invention constituent (c) has a hydrophilic-lipophilic-balance (HLB) value of about 4 or higher.

More typically, in compositions or products according to the present invention constituent (c) has a hydrophilic-lipophilic-balance (HLB) value of about 5 or higher.

More typically, in compositions or products according to the present invention constituent (c) has a hydrophilic-lipophilic-balance (HLB) value of about 6 or higher.

In certain compositions and products of the present invention, constituent (c) comprises or consists of one or more non-ionic surfactants selected from the group consisting of (poly) alkoxylated alcohols, (poly) alkoxylated phosphate esters and (poly) alkoxylated tristyrylphenols.

Said (poly) alkoxylated phosphate esters are phosphate esters which are substantially in free acid form, i.e. not in salt form. Thus, said (poly) alkoxylated phosphate esters have not been neutralized.

In certain compositions and products of the present invention, constituent (c) comprises or consists of one or more non-ionic surfactants selected from the group consisting of (poly) alkoxylated linear saturated or monounsaturated C₁₂-C₁₈-alcohols and (poly) alkoxylated phosphate esters of linear saturated or monounsaturated C₁₂-C₁₈-alcohols.

In various compositions and products of the present invention, constituent (c) comprises or consists of one or more non-ionic surfactants having a degree of alkoxylation in the range of from about 2 to about 14.

In other compositions and products of the present invention, constituent (c) comprises or consists of one or more non-ionic surfactants having a degree of alkoxylation in the range of from about 4 to about 10.

In still other compositions and products of the present invention, constituent (c) comprises or consists of one or more non-ionic surfactants selected from the group consisting of (poly) ethoxylated linear saturated or monounsaturated C₁₂-C₁₈-alcohols and (poly) ethoxylated phosphate esters of linear saturated or monounsaturated C₁₂-C₁₈-alcohols.

In other compositions and products of the present invention, constituent (c) comprises or consists of one or more non-ionic surfactants comprising from to about 2 to about 14 ethylene glycol units (PEG-2 to PEG-14).

In other compositions and products of the present invention, constituent (c) comprises or consists of one or more non-ionic surfactants comprising from about 4 to about 10 ethylene glycol units (PEG-4 to PEG-10).

In other compositions and products of the present invention, constituent (c) comprises or consists of one or more non-ionic surfactants comprising selected from the group consisting of tristyrylphenol ethoxylates.

In other compositions and products of the present invention, constituent (c) comprises or consists of one or more non-ionic surfactants comprising selected from the group consisting of tristyrylphenol ethoxylates comprising from about 6 to about 80 ethylene glycol units (PEG-6 to PEG-80).

In still other compositions and products of the present invention, constituent (c) comprises or consists of one or more non-ionic surfactants comprising selected from the group consisting of tristyrylphenol ethoxylates comprising from about 10 to about 60 ethylene glycol units (PEG-10 to PEG-60).

In certain compositions and products of the present invention, the total amount of constituent (c) is in the range of from about 2% by weight to 20% by weight, based on the total weight of the composition.

In certain compositions and products of the present invention, constituent (c) comprises or consists of one or more non-ionic surfactants selected from the group consisting of ethoxylated linear saturated or monounsaturated C₁₂-C₁₈-alcohols with about 4 to about 10 ethylene glycol units (PEG-4 to PEG-10), and wherein the total amount of constituent (c) is in the range of from about 4% by weight to about 20% by weight, based on the total weight of the composition.

In various compositions and products of the present invention, constituent (c) comprises or consists of one or more non-ionic surfactants selected from the group consisting of ethoxylated phosphate esters of linear saturated or monounsaturated C₁₂-C₁₈-alcohols with about 4 to about 10 ethylene glycol units (PEG-4 to PEG-10), and wherein the total amount of constituent (c) is in the range of from about 2% by weight to about 10% by weight, based on the total weight of the composition.

Suitable linear alcohol ethoxylates as constituent (c) of compositions and products of the present invention are commercially available from many sources such as Shell Chemical (Neodol™), Dow (Tergitol™), or Croda (Brij™ and Synperonic™), and BASF (Lutensol™).

These surfactants are co-incorporated into the same urea matrix as the active ingredient (constituent (a)). One function of the non-ionic surfactant(s) of constituent (c) of compositions or products of the present invention is to stabilize the urea complex structure and the non-ionic surfactant(s) are incorporated the urea complex, leading to harder compositions or products which are more readily handled, and for example, allow better extrusion.

It has been found that while oleyl and other unsaturated alcohol ethoxylates can be used effectively in the context of the present invention, ethoxylated linear saturated alcohols containing 12 to 18 carbons (C₁₂-C₁₈) are suitable by virtue of the desired (superior) hardness of the resulting composition or product of the present invention.

A second function of the non-ionic (neutral) surfactants of constituent (c) is to provide wetting when the composition or product of the present invention is added to water. Particularly suitable surfactants which provide effective wetting and a harder extruded composition or product are C12-C18 linear saturated alcohols functionalized with a PEG-5 to PEG-10 chain. Examples of this particularly suitable class are Synperonic™ 13/6 (tridecyl alcohol PEG-6, Croda), Synperonic™ A7 (C12-15 alcohol, PEG-7, Croda) and Lutensol™ TDA 6 (tridecyl alcohol PEG-6, BASF).

For certain actives, such as actives which are stabilized by the presence of acid, the non-ionic surfactant typically includes an acidic group which is not neutralized. Phosphate ester surfactants in the acid form are suitable for this purpose since they are readily incorporated into the urea matrix together with the active. Phosphate ester surfactants are well-known in the art and are commercially available, for example from BASF and Croda. Polyethylene glycol (PEG) chains may be preferred over mixed chains of polyethylene glycol and polypropylene glycol. Agnique™ PE TDA 9 (tridecyl phosphate PEG-9, BASF) and Crodafos™ O5A (oleyl phosphate PEG-5, Croda) are examples of suitable phosphate ester surfactants of constituent (c) for this aspect of the present invention.

These phosphate ester surfactants in the acid form (i.e. without neutralization) are co-incorporated in urea in the composition or products of the present invention, and enhance the stability of certain pH-sensitive actives, such as triketone herbicides like tembotrione or mesotrione. The stability of these active is improved by the incorporation of phosphate ester surfactants in the acid form, as further described in the Examples.

As also described in the Examples, incorporation of certain acids such as phosphoric acid (may be used as a liquid (e.g. 85% in water) or as solid) or methanesulfonic acid (may be used as a liquid (e.g. 70% in water) or as solid) into the composition or product of the present invention is an effective means to stabilize pH-sensitive active ingredients such as mesotrione, tembotrione, or isoxaflutole. Phosphoric acid preferred to be incorporated into compositions or products of the present invention comprising active ingredients requiring low pH for chemical stability, such as mesotrione, tembotrione, or isoxaflutole. The amount of these acids depends extent on the amount of active ingredients present in the compositions or products of the present invention as well as the amounts and types of further constituents therein. Generally, the amount of phosphoric acid or methanesulfonic acid is in the range of about 0.3 wt. % to about 1.5 wt. %, typically in the range of about 0.6 wt. % to about 1.2 wt. %, in each case based on the total amount of the composition or product of the present invention.

Optionally, the compositions and products of the present invention comprise as constituent (d) one or more polymeric dispersants different from constituent (c).

Additional embodiments of the present invention comprise hydrolysis of the urea complex in the presence of a water-soluble dispersant polymer. Suitable polymer concentrations in the hydrolysis mixture are 0.1% to 0.6% on a soluble basis. It has been found that urea complexes of certain actives form small particles with enhanced bioavailability when hydrolysis is conducted in solutions of suitable polymeric dispersants. As described in the Examples, prothioconazole and tembotrione urea complexes for example are particularly suitable for such embodiments. Amphiphilic water-soluble polymers such as Sokalan™ CP 9 (BASF) and Geropon™ TA/72 (Solvay) are suitable water-soluble polymeric dispersants.

Typically, one or more dispersants are added to stabilize particle suspension. Polymeric dispersants such as Sokalan™ CP 9 (BASF), Atlox™ 4914 and Atlox™ 4915 (Croda) and Break-thru® DA-647 (Evonik) are suitable dispersants for this aspect of the invention as are lignosulfonates such as those sold by Borregaard and Ingevity.

A further aspect of the invention is particularly applicable to urea complexes prepared by slow cooling, as described below and in the Examples. Compositions and formulations prepared by slow cooling tend to form larger active particles upon hydrolysis of the urea complex. Thus, incorporation of dispersants into the compositions or formulations of the present invention can be beneficial in maintaining suspension of the active particles in water. Typically, lignosulfonate dispersants in powder form are dry-blended with the urea complex in amounts of about 10% to about 30% by weight. These classes of dispersants are well-known in the art for maintaining particulate suspension and suppressing agglomeration. Particularly suitable dry powder dispersants for this aspect of the invention are Agnique™ DDL (BASF), and Ultrazine™ NA (Borregaard).

If present in a composition or product according to the invention, constituent (d) comprises or consists of one or more polymeric dispersants different from constituent (c), wherein constituent (d) is selected from the group consisting of polycarboxylates and salts thereof, maleic anhydride-isobutylene copolymers and salts thereof, and (block) copolymers of styrene oxide and ethylene oxide, lignosulfates, and mixtures thereof.

If present in a composition or product according to the invention, constituent (d) comprises or consists of one or more polymeric dispersants different from constituent (c), wherein constituent (d) more typically is selected from the group consisting of polycarboxylates and sodium salts, maleic anhydride-isobutylene copolymers and sodium salts thereof and (block) copolymers of styrene oxide and ethylene oxide, and mixtures thereof.

Particularly suitable polymeric dispersants for use in the context of the present invention are for example Geropon® TA/72 (Solvay), a water-soluble sodium polycarboxylate, Sokalan® CP 9 (BASF), a copolymer of maleic acid and an olefin as the sodium salt, and/or Break-thru® DA-647 (Evonik), a non-ionic modified copolymer of styrene oxide and ethylene oxide.

Typically, the polymeric dispersants of constituent (d) have a molecular weight Mn of about 1000 g/mol or higher, for example in the range of from about 1000 g/mol to about 200000 g/mol.

If present in a composition or product according to the invention, the total amount of constituent (d) typically is in the range of about 1 to about 15% by weight, based on the total weight of the composition or product.

If present in a composition or product according to the invention, the total amount of constituent (d) more typically is in the range of about 2 to about 12% by weight, based on the total weight of the composition or product.

If present in a composition or product according to the invention, the total amount of constituent (d) more typically is in the range of about 3 to about 9% by weight, based on the total weight of the composition or product.

Optionally, the compositions and products of the present invention comprise as constituent (e) one or more wetting agents different from constituents (c) and (d).

Suitable examples of such wetting agents (wetters) include ethoxylates of branched alcohols, particularly Guerbet alcohol ethoxylate marketed as Lutensol™ XL50-XL80 by BASF. Alkyl polyglucosides such as Agnique™ PG 8105 and Agnique™ PG 264 (BASF) are also suitable wetters, but these materials are supplied in water, and therefore tend to lead to a softer rheology of the composition or product.

Thus, effective wetters which are commercially available in the form of a dry powder, but which dissolve readily in water, are particularly suitable in this aspect of the invention. Examples of particularly suitable dry wetters with nonlinear structure are Morwet™ D-425 (alkylnaphthalene sulfonate condensate, Nouryon) and Agnique™ ANS 3DNPW (BASF). Another example of a particularly suitable wetter is Morwet™ EFW (alkylnaphthalene sulfonate condensate, Nouryon).

If present in a composition or product of the invention, constituent (e) typically comprises or consists of one or more wetting agents with a non-linear structure, wherein constituent (e) is different from constituents (c) and (d).

If present in a composition or product of the invention, constituent (e) comprises or consists of one or more wetting agents different from constituents (c) and (d), wherein constituent (e) typically is selected from the group consisting of Guerbet alcohol ethoxylates, alkyl polyglucosides, alkylnaphthalene sulfonate condensates, and mixtures thereof.

If present in a composition or product of the invention, constituent (e) comprises or consists of one or more wetting agents different from constituents (c) and (d), wherein constituent (e) more typically is selected from the group consisting of ethoxylated branched alcohols.

If present in a composition or product of the invention, constituent (e) comprises or consists of one or more wetting agents different from constituents (c) and (d), wherein constituent (e) more typically is selected from the group consisting of alkylnaphthalene sulfonate condensates.

If present in a composition or product according to the invention, the total amount of constituent (e) typically is in the range of about 5 to about 30% by weight, based on the total weight of the composition or product.

If present in a composition or product according to the invention, the total amount of constituent (e) typically is in the range of about 5 to about 20% by weight, based on the total weight of the composition or product.

Optionally, the compositions and products of the present invention comprise as constituent (f) one or more water-soluble polymeric binders different from constituents (c), (d) and (e).

In certain embodiments, urea complexes of the present invention are typically prepared from the melt. The constituents of the melt can be prepared by mixing in an open or closed vessel, by circulation in an extruder, the use of a sigma mixer or planetary mixer or any other common mixing technique known in the art provided that temperature can be controlled. In case the composition or product according to the present invention is obtained by extrusion with mixing it may be advantageous to add one or more surfactants first to provide lubrication.

If water is added to reduce the melting point of the mixture it is typically less than 10% by weight of the overall mass of the composition. An aqueous solution of water-soluble polymers which can serve as binders can be used, particularly when extruding.

If present in a composition or product of the invention, constituent (f) typically comprises or consists of one or more water-soluble polymeric binders different from constituents (c), (d) and (e) selected from the group consisting of cellulose ethers and salts thereof.

If present in a composition or product of the invention, constituent (f) typically comprises or consists of one or more water-soluble polymeric binders different from constituents (c), (d) and (e) selected from the group consisting of methylcellulose polymers, water-soluble hydroxypropyl methylcellulose, salts thereof, and mixtures thereof.

Cellulose ethers suitable as water-soluble polymeric binders of constituent (f) are for example available under the trade name Methocel™ from International Flavors and Fragrances. Methocel™ J12 MS is a particularly suitable binder in the context of the present invention.

If present in a composition or product according to the invention, the total amount of constituent (f) typically is in the range of about 0.5 to about 3% by weight, based on the total weight of the composition or product.

In certain compositions or products of the present invention, the total amount of methanol in the composition or product is less than about 1% by weight, based on the total weight of the composition or product.

In certain compositions or products of the present invention, the total amount of ethanol in the composition or product is less than about 1% by weight, based on the total weight of the composition or product.

In other compositions or products of the present invention, the total amount of C₁-C₆ alcohols in the composition or product is less than about 1% by weight, based on the total weight of the composition or product.

In certain compositions or products of the present invention, the total amount of oleic acid in the composition or product is less than about 1% by weight, based on the total weight of the composition or product.

In various compositions or products of the present invention, the total amount of constituents in salt form in the composition or product is about 10% by weight or less, based on the total weight of the composition or product.

In other compositions or products of the present invention, the total amount of constituents in salt form in the composition or product is about 5% by weight or less, based on the total weight of the composition or product.

Thus, the compositions or products of the present invention typically are substantially free of constituents in salt form.

If constituent (a) comprises or consists of one or more pesticidal active ingredients, these can be selected from the group consisting of pesticidal active ingredients that meet the structural and physical criteria defined in the context of the present invention.

Typically, constituent (a) of compositions, products and application mixtures according to the present invention comprise or consist of one or more pesticidal active ingredients, wherein constituent (a) typically comprises or consists of one or more active ingredients selected from the group consisting of fungicides, herbicides, insecticides, nematicides, acaricides, molluscicides, bactericides, and safeners.

Pesticidal active (pesticides) and safeners that may be used as constituent (a) of compositions, products and application mixtures according to the present invention and the common names used herein are known in the art, see, for example, “The Pesticide Manual” 16^{th} Edition, British Crop Protection Council 2012; these include—provided the criteria for constituent (a) defined in the context of the present invention are fulfilled—the known stereoisomers (in particular racemic and enantiomeric pure isomers) and derivatives such as salts or esters, and particularly the commercially customary forms. Where a pesticide, is referenced generically herein by its common name, unless otherwise restricted, that pesticide includes all forms known in the art such as salts, esters, free acids and free bases, as well as stereoisomers thereof.

If constituent (a) comprises or consists of one or more herbicides, the one or more herbicides can be selected from the group consisting of acetyl CoA carboxylase (ACCase) inhibitors, enolpyruvyl shikimate-3-phosphate synthase (EPSPS) inhibitors, glutamine synthetase inhibitors, auxins, photosystem I (PS I) inhibitors, photosystem II (PS II) inhibitors, acetolactate synthase (ALS) or acetohydroxy acid synthase (AHAS) inhibitors, mitosis inhibitors, protoporphyrinogen oxidase (PPO) inhibitors, 4-Hydroxyphenylpyruvate dioxygenase (HPPD) inhibitors, cellulose inhibitors, oxidative phosphorylation uncouplers, dihydropteroate synthase inhibitors, fatty acid and lipid biosynthesis inhibitors, auxin transport inhibitors and carotenoid biosynthesis inhibitors, salts and esters thereof, racemic mixtures and resolved isomers thereof, and mixtures thereof.

If constituent (a) comprises or consists of one or more insecticides, the one or more insecticides can be selected from the group consisting of organo(thio)phosphates, carbamates, pyrethroids, insect growth regulators, chitin synthesis inhibitors, insect steroid hormone antagonists, juvenoids, lipid biosynthesis inhibitors, nicotinic acetylcholine receptor disruptors, allosteric modulators, bioinsecticides, GABA antagonist compounds, mitochondrial electron transport inhibitors, uncouplers, oxidative phosphorylation inhibitors, moulting disruptors, oxidase inhibitors, sodium channel blockers, ryanodine receptor inhibitors, esters thereof, racemic mixtures and resolved isomers thereof, and mixtures thereof.

If constituent (a) comprises or consists of one or more fungicides, the one or more fungicides can be selected from the group consisting of respiration inhibitors (such as inhibitors of complex II, inhibitors of complex III), sterol biosynthesis inhibitors (such as C14 demethylase inhibitors, delta 14-reductase inhibitors, inhibitors of 3-keto reductase), nucleic acid synthesis inhibitors (such as phenylamides or acyl amino acid fungicides, other nucleic acid inhibitors), inhibitors of cell division and cytoskeleton (such as tubulin inhibitors or other cell division inhibitors), inhibitors of amino acid and protein synthesis (such as methionine synthesis inhibitors, protein synthesis inhibitors), signal transduction inhibitors (MAP/histidine kinase inhibitors, G protein inhibitors), lipid and membrane synthesis inhibitors (such as phospholipid synthesis inhibitors, lipid peroxidation inhibitors, phospholipid biosynthesis and cell wall deposition inhibitors, acid amide hydrolase inhibitors), inhibitors with multi-site action, cell wall inhibitors (such as glucan synthesis inhibitors, melanin synthesis inhibitors), plant defense inducers, racemic mixtures and resolved isomers thereof, and mixtures thereof.

Safeners in the context of the present invention typically are herbicide safeners. Typically, safeners are selected from the group consisting of benoxacor, cloquintocet and agriculturally acceptable esters thereof, cyometrinil, cyprosulfamide, dichlormid, dicyclonon, dietholate, fenchlorazole and agriculturally acceptable esters thereof, fenclorim, flurazole, fluxofenim, furilazole, isoxadifen and agriculturally acceptable esters thereof, mefenpyr and agriculturally acceptable esters thereof, mephenate, metcamifen, naphthalic anhydride, oxabetrinil, and mixtures thereof.

In the context of the present invention, constituent (a) typically comprises or consists of pesticidal actives selected from the group consisting of chlorotriazine herbicides, pyridine fungicides, aroylcyclohexanedione herbicides, carbanilate herbicides, conazole fungicides, pyridylpyrazole insecticides, and mixtures thereof.

In the context of the present invention, constituent (a) more typically comprises or consists of actives selected from the group consisting of atrazine, cyprosulfamide, fluopyram, isoxaflutole, mesotrione, phenmedipham, prothioconazole, tembotrione, tetraniliprole, and mixtures thereof.

In certain embodiments of the present invention, constituent (a) comprises or consists of tembotrione, mesotrione, phenmedipham, or combinations thereof.

In other embodiments of the present invention, constituent (a) comprises or consists of fluopyram.

In other embodiments of the present invention, constituent (a) comprises or consists of tetraniliprole.

If constituent (a) comprises or consists of one or more pharmaceutical active ingredients, these can be selected from the group consisting of pharmaceutical active ingredients that meet the structural and physical criteria defined in the context of the present invention.

Suitable pharmaceutical active ingredients are for example blood thinners that may lower the risk of stroke, deep vein thrombosis (DVT), pulmonary embolism (PE), and similar conditions, such as Rivaroxaban.

Suitable pharmaceutical active ingredients are for example anti-cancer and anti-angiogenic agents like Regorafenib that possesses various activities including inhibitory activity on the VEGFR, PDGFR, raf, p38, and/or flt-3 kinase signalling molecules and it can be used in treating various diseases and conditions like hyper-proliferative disorders such as cancers, tumors, lymphomas, sarcomas and leukemias.

Other suitable pharmaceutical active ingredients are for example dihydropyridine type calcium antagonists like nitrendipine, nimodipine or lacidipine. Nimodipine for example is an antihypertensive drug that can reduce brain damage caused by bleeding from a burst blood vessel.

In a further aspect, the present invention relates to products obtainable or obtained by extrusion of a composition as defined in the context of the present invention, wherein the extrusion temperature typically is in the range of from about 85° C. to about 110° C., and subsequent cooling of the molten composition to a temperature below 55° C.

In a further aspect, the present invention relates to products obtainable or obtained by melting a composition as defined in the context of the present invention, and subsequent cooling of the molten composition to a temperature below 55° C., wherein typically melting of the composition is performed such that the temperature of the molten composition is in the range of from about 75° C. to about 110° C.

The products according to the present invention typically contain methanol and/or ethanol in a total amount of less than about 1% by weight, based on the total weight of the product.

The products according to the present invention typically contain C₁-C₆ alcohols in a total amount of less than about 1% by weight, based on the total weight of the product.

The products according to the present invention typically contain oleic acid in a total amount of less than about 1% by weight, based on the total weight of the product.

In a further aspect, the present invention relates to formulations comprising a composition or product as defined in the context of the present invention, additionally comprising one or more further constituents selected from the group consisting of adjuvants, liquid ingredients at 25° C. and 1013 mbar, solid active ingredients at 25° C. and 1013 mbar, and mixtures thereof.

Said adjuvants typically are selected from the group of substances well-known as adjuvants in the art of agricultural formulation. Adjuvants that may be included into formulations of the present invention are for example antifreeze agents, such as alkylene glycols. Suitable antifreeze agents are glycerol or propylene glycol, typically included in an amount in the range of from about 3% by volume to about 20% by volume, typically in the range of from about 5% by volume to about 15% by volume.

A formulation of the present invention typically is a formulation selected from the group consisting of wettable granules (WG), wettable powders (WP), and oil dispersions (OD).

If a formulation of the present invention is an oil dispersion (OD) formulation, the one or more formulation adjuvants typically are selected from the group consisting of oily active ingredients, vegetable oils, oily solvents, and mixtures thereof.

In one embodiment, the formulation of the present invention is an oil dispersion (OD) formulation, comprising an oily solvent, wherein the oily solvent comprises or is a long-chain fatty acid methyl ester, typically methyl soyate (soy methyl esters).

In another embodiment, the formulation of the present invention is an oil dispersion (OD) formulation, comprising an oily herbicide, wherein the oily herbicide comprises or is acetochlor.

In some embodiments, the formulation of the present invention is a wettable granule (WG) formulation or a wettable powder (WP) formulation, wherein the adjuvant comprises or is an emulsifier powder or a powder dispersant.

In some embodiments, the formulation of the present invention is a wettable granule (WG) formulation or a wettable powder (WP) formulation, wherein the adjuvant comprises or is a dry emulsifier powder or a dry powder dispersant.

Another aspect of the invention, described in more detail in Example 1 below, is a method for the preparation of urea complexes of the present invention by evaporation of an alcohol solution. This method is particularly suited to laboratory-scale preparations without specialized equipment such as an extruder or spray drier. In this process, urea, the active, and an organic compound, typically containing 16 to 65 carbons, are combined and fully dissolved in an alcohol solution. Examples of suitable organic compounds are methyl soyate and ethoxylates of C₁₂ to C₁₈ alcohols. Macol™ CSA 20 (cetyl-stearyl alcohol PEG-20, BASF) is also a suitable organic compound for this embodiment.

A further method of obtaining the compositions of the present invention is exemplified in Example 3 below. The active is dissolved in a mixture of water, urea and non-ionic surfactant at an elevated temperature, typically in the range of from about 80° C. to about 110° C. When dissolution of the active is complete, the active ingredient-urea mixture is added to an oily diluent at the same temperature as the urea solution (or higher) with rapid stirring. Stir speed and oil volume are typically sufficient to provide a fine emulsion of the urea mixture. The oily diluent is typically a hydrocarbon with a branched structure in order to minimize solubility in urea with a boiling point at least 10° C. above the temperature of the operation. Isooctane is a particularly suitable oily diluent for this embodiment. Typically, an emulsifier with a branched structure and an HLB below 14 is added to the oily diluent. Tween™ 61 (sorbitan stearate PEG-4, HLB 10, Croda) is a suitable emulsifier for this process. The urea complex of the present invention can be isolated by filtration.

In a further aspect, the present invention relates processes for preparing a composition as defined in the context of the present invention, characterized by the following steps:
1. Combining constituents (a), (b), (c), and optionally one or more further constituents selected from the group consisting of water, and constituents (d), (e) and (f) as defined hereinabove,
2. Mixing the combination resulting from step 1 at a temperature of in the range of from about 60° C. to about 140° C., typically at a temperature of in the range of from about 75° C. to about 125° C., more typically at a temperature of in the range of from about 80° C. to about 110° C.,
3. Cooling the composition resulting from step 2.

The temperature in step 3 is lower than the temperature in step 2 to an extent such that at least a semi-solid product is obtained which upon further cooling solidifies into a composition according to the present invention.

The mixing in step 2 can for example be performed with a stirrer, a twin-screw mixer or other suitable methods known in the art.

Step 3 includes extrusion, spray drying, spray chilling, prilling, spheronization, combinations thereof or other means known in the art.

In a further aspect, the present invention relates to a product obtainable or obtained by the above-defined process, wherein, upon adding the product at 25° C. to an amount of water sufficient to dissolve the urea, the one or more active ingredients of constituent (a) is substantially present as nanoparticles with diameters substantially below 100 nm, as determined by dynamic light scattering,

In a further aspect, the present invention relates to a product obtainable or obtained by the above-defined process, wherein, upon adding the product at 25° C. to an amount of an aqueous emulsifier dilution sufficient to dissolve the urea, an emulsion of the liquid one or more active ingredients of constituent (a) is obtained which is stable against crystallization of said one or more the active ingredients of constituent (a) for at least 30 minutes at 25° C.

In a further aspect, the present invention relates to an application mixture, comprising
a composition or product of the present invention as defined in hereinabove,
water in an amount by weight at least 10 times, typically at least 25 times, more typically at least 50 times, the amount of said composition or product, and
one or more constituents selected from the group consisting of further adjuvants, other diluents, and other active ingredients.

In certain embodiments, the application mixture is a spray application mixture.

In other embodiments, the application mixture is an agricultural spray application mixture.

In further aspects, the present invention relates to a composition, product or application mixture as defined in the context of the present invention, comprising a pesticidal active ingredient, for use in a method of controlling undesired vegetation, plant pests, (phytopathogenic) fungi or (phytopathogenic) nematodes.

In further aspects, the present invention relates to a method for controlling undesired vegetation, plant pests, phytopathogenic fungi or phytopathogenic nematodes comprising the application of a composition, product or application mixture defined in the context of the present invention, comprising a pesticidal active ingredient, to said undesired vegetation, plant pests, phytopathogenic fungi or phytopathogenic nematodes.

In further aspects, the present invention relates to the use of the composition, product or application mixture defined in the context of the present invention, comprising a pesticidal active ingredient, to control undesired vegetation, plant pests, phytopathogenic fungi or phytopathogenic nematodes.

In the context of the present invention, phytopathogenic nematodes are in particular plant parasites. Most nematodes feeding on higher plants are obligatory parasites. Economically high impact plant parasites are for example a root knot, cyst and lesion nematodes.

In further aspects, the present invention relates to the composition, product or application mixture defined in the context of the present invention, comprising a pharmaceutical active ingredient, for use as a medicament.

In further aspects, the present invention relates to the composition, product or application mixture defined in the context of the present invention, comprising a pharmaceutical active ingredient, for use in a method of treating an animal or human body.

In further aspects, the present invention relates to a method of treating a subject in need thereof comprising (typically orally) administering a pharmaceutically effective amount of the composition, product or application mixture defined in the context of the present invention, comprising a pharmaceutical active ingredient, to said subject.

In further aspects, the present invention relates to the use of the composition, product or application mixture defined in the context of the present invention, comprising a pharmaceutical active ingredient, in the treatment of an animal or human body, wherein said composition, product or application mixture typically is orally administered to said animal or human body.

In a further aspect, the present invention relates to an application mixture (known as tank-mix in the case of agricultural active ingredients) obtainable or obtained by diluting a composition. product or formulation of the present invention with an appropriate amount of water, wherein typically the ratio by weight of water to composition, product or formulation is in the range of from about 200:1 to about 10:1, typically in the range of from about 100:1 to about 20:1.

Such as application mixture may comprise one or more further additives, formulation adjuvants and/or pesticides.

In a further aspect, the present invention relates to methods of making an application mixture of the present invention, characterized in that a composition, product or formulation is poured (slowly) into a water contained vessel under (mild) agitation, optionally including one or more further additives, formulation adjuvants and/or pesticides into the application mixture.

Typically, in such methods of making an application mixture of the present invention the ratio by weight of water to composition, product or formulation of the present invention is in the range of from about 200:1 to about 10:1, typically in the range of from about 100:1 to about 20:1.

The spray application mixture of the present invention may be applied to a field or an area of application according to practices known to those skilled in the art. A spray application mixture according to the present invention may be applied to the field at different stages of the crop plant, depending on the pesticidal active ingredient(s) present in or as constituent (a). In some embodiments, the spray application mixture is applied to the soil, before planting the crop plants or after planting the crop plants, but pre-emergent to the crop plants. In other embodiments, the spray application mixture is applied post-emergent to the crop plant.

Preferred crop plants in the context of the present invention are corn, soybean, cotton, wheat, oilseed rape, canola, and sugarbeet. The compositions, products and spray application mixtures of the present invention may also be applied to other useful plants, such as vines, trees (for example palm trees), fruits (for example apples, pears, prunes, bananas etc.), nuts (for example almonds, pecans, peanuts etc.), or vegetables (for example tomatoes, dry beans, snap beans, potatoes etc.).

Compositions, products or application mixtures of the present invention comprising one or more herbicides as (part of) constituent (a) are useful for controlling a wide variety of weeds, i.e., plants that are considered to be a nuisance or a competitor of commercially important crop plants, such as corn, soybean, cotton, wheat, oilseed rape, canola, sugarbeet, dry beans, snap beans, and potatoes etc. In some embodiments, the application mixtures are applied before the weeds emerge (i.e., pre-emergence application).

Monocotyledonous weeds belong, for example, to the genera Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristylis, Sagittaria, Eleocharis, Scirpus, Paspalum, Ischaemum, Sphenoclea, Dactyloctenium, Agrostis, Alopecurus and Apera.

Dicotyledonous weeds belong, for example, to the genera Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Kochia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum and Euphorbia.

Having described the invention in detail, it will be apparent that modifications and variations are possible without departing from the scope of the invention defined in the appended claims.

### EXAMPLES

The following non-limiting examples are provided to further illustrate the present invention.

Unless indicated otherwise, all amounts and percentages are by weight.

### Abbreviations and Materials Used

PEG=Polyethylene glycol
Macol® CSA 20=PEG-20 ether of cetostearyl (cetyl/stearyl) alcohol=Ceteareth-20 (BASF) MTBE=methyl-t-butyl ether
Klearfac® AA 270=Phosphate ester of polyoxyalkylated fatty alcohol [Oxirane, 2-methyl-, polymer with oxirane, mono-C₁₀-16-alkyl ethers, phosphates, CAS No. 68649-29-6], 85% active, and 15% phosphoric acid (BASF)
Lutensit® AE-P=Phosphate ester of an ethoxylated/propoxylated medium chain alcohol (BASF)
Crodafos™ SG=PEG-10 PPG-5 Cetyl Phosphate=PPG-5-Ceteth-10 Phosphate (Croda)
Phospholan™ PS-220=C₁₀-14 Alcohol (30 EO) Ethoxylate 104-Phosphate Ester (Nouryon)
Brij® O20=Polyoxyethylene (20) Oleyl Ether=Oleth-20 (Croda)
Crodafos™ O10A=Complex ester of phosphoric acid and ethoxylated cosmetic grade oleyl alcohol (Croda)
Tween 61®=Polyoxyethylene (4) sorbitan monostearate=sorbitan stearate PEG-4 (Croda)
Ultrazine™ NA=Sodium Lignosulphonate dispersant (Borregaard) rpm=revolutions per minute
Methocel™ J12 MS=Surface treated hydroxypropyl methylcellulose (HPMC) based polymer (International Flavors and Fragrances), typically used as 2% aqueous solution
Pluronic® P123=Ethylene oxide-propylene oxide block copolymer (BASF)
Synperonic™ A7=An ethoxylate of a fully saturated C₁₂-C₁₅ alcohol. This water-soluble non-ionic surfactant is an effective wetting agent and detergent (Croda)
Break-thru® EM O7=Oleyl alcohol PEG-7 (Evonik)
Agnique® DDL=Dispersant based on lignosulfonate (BASF)
Toximul® 8244=PEG-16 Polyethoxylated castor oil (Stepan)
Pluronic® P123=Poly (ethylene glycol)-block-poly (propylene glycol)-block-poly (ethylene glycol) (Sigma Aldrich)
Agnique® PE TDA 9=Tridecyl alcohol phosphate ester, PEG-9 (BASF)
Agnique® ANS 3DNPW=Blend of alkyl naphthalene sulfonate (BASF), wetting agent
Synperonic™ 13/6=Polyoxyethylene (6) isotridecanol (Croda)
Sokalan®=Sokalan® CP 9=Maleic acid-olefin copolymer, 25% aqueous solution (BASF)
Morwet® EFW=Sodium alkyl naphthalene sulfonate blend dispersant (Nouryon)
AAtrex® Nine-O®=88.2% Atrazine, 1.8% related compounds, 10% other ingredients (Syngenta)
Laudis®=34.5% Tembotrione, 65.5% other ingredients (Bayer)
Callisto®=40% Mesotrione, 60% other ingredients (Syngenta)
Proline®=41% Prothioconazole, 59% other ingredients (Bayer)
Urea=99% urea (Fisher Scientific)
Haake™ Mini CTW=hot melt extruder, ThermoFisher Scientific
Malvern Nano-ZS Zetasizer=Dynamic light scattering equipment
Break-thru® S233: Biodegradable trisiloxane surfactant (Evonik)
Soprophor® FLK=Anionic tristyrylphenol ethoxylate phosphate ester emulsifier, aqueous solution at about 40% active (Solvay)
Lutropur® MSA=70% aqueous solution of methanesulfonic acid (BASF)
Geropon® TA/72=Sodium polycarboxylate, CAS No. 223571-91-3, anionic surfactant, polymeric dispersant (Solvay)
Makon® TSP-60=Tristyrylphenol ethoxylate PEG-60 (Stepan)
Break-thru® DA-647=Non-ionic modified copolymer of styrene oxide and ethylene oxide (Evonik)
Myrj™ S8=Polyoxyl 8 stearate=PEG-8 Stearate (Croda)
Steposol® OE=Methyl oleate
Stepwet® DF-95=Sodium lauryl sulfate (93%) (Stepan), dry powder
Dowfax® 8390=Diphenylether disulfonate, sodium salt, C16 alkylate (Dow Chemical)
Dowfax® 3B2=Diphenylether disulfonate, sodium salt, C10 alkylate (Dow Chemical)
Stepfac™ 8181=Polyethylene glycol (6) tridecyl ether phosphate
Makon® TSP-12=Tristyrylphenol ethoxylate PEG-12 (Stepan)
Toximul® 8320=Butyl-based ethylene oxide-propylene oxide block copolymer (Stepan) (average MW 5500, HLB 12)
Makon® TD-12=Tridecyl alcohol ethoxylate, PEG-12 (Stepan)
Crodafos® T6A=Tridecyl phosphate ester, PEG-6 (Croda)
Soprophor® CY/8=A nonionic tristyrylphenol ethoxylate emulsifier (CAS No. 99734-09-05);
HLB: 13.5, paste form (Solvay)
Soprophor® S-25/80=A nonionic tristyrylphenol ethoxylate; HLB: 14.5; about 80% active in water, liquid (Solvay)

In the Examples that follow, compositions of the present invention sometimes are also referred to as complexes

### EXAMPLES 1-11

### Example 1: Preparation of Urea Complexes of Atrazine by the Evaporation Method

The following components, as shown in Table 1, were combined in a bottle and placed in an oven at 55° C. The bottle was removed from the oven, stirred, and returned to the oven to complete dissolution.

TABLE 1
Components used to form urea complexes
of Atrazine by the evaporation method

| Ingredient | Amount | |
|---|---|---|
| Atrazine | 8.0 | g |
| Urea | 60 | g |
| Macol ® CSA 20 | 2.0 | g |
| Methanol | 350 | mL |

7.0 g of Vistive® soy methyl esters was added to the bottle after dissolution was complete. The bottle weas shaken and the contents transferred to a pear-shaped flask and the solvent evaporated on a rotary evaporator until white precipitate began to form, which occurred abruptly during evaporation. The precipitate was recovered by filtration. The complex exhibited rapid and near-complete precipitation, with only a minor amount of complex forming in the filter flask, that was not recovered. The product was rinsed with MTBE and dried overnight at 55° C. under 24″ Hg (about 0.81273 bar) vacuum with nitrogen purge. 51.8 g of the product, a white powder, was recovered which was found to contain 9.9% atrazine and 10.7% total triazine compounds. When added to water, the urea dissolved leaving fine atrazine particles suspended in a cloudy solution.

### Example 2: Preparation of Urea Complexes of Mesotrione and a Phosphate Ester Surfactant by the Evaporation Method

In four different 4-oz (120 mL) bottles, each 15 g of urea, 80 mL of methanol, and 0.5 g Brij® O20 were combined in capped. The bottles were placed in an oven held at 55° C. with occasional swirling until the contents had fully dissolved. The bottles were removed from the oven and a stirring bar added along with 1.6 g of mesotrione and 0.5 g of the respective phosphate ester shown in Table 2. The phosphate ester in this case not only acts in the formation of the urea complex, but also in stabilizing the mesotrione against decomposition due to the acidity of the un-neutralized phosphate ester. The bottles were stirred briefly at room temperature until the mesotrione had dissolved (approximately five minutes). The respective bottle was swirled and the contents transferred to a roundbottom flask, and 1.8 g of soy methyl esters was added to the flask. The solvent was evaporated on a rotary evaporator until white precipitate began to form in an exothermic step accompanied by accelerated methanol evaporation, which, after an abrupt start, occurred rapidly. The precipitate was transferred to a Buchner funnel with a coarse frit. The filtrate was yellow-brown. Remaining product in the flask was rinsed into the Buchner funnel with methyl-t-butyl ether and vacuum applied. The product was a fine white powder which retained a fair amount of solvent even after vacuum filtration. It was dried overnight at 55° C. under 24″ Hg (about 0.81273 bar) vacuum with nitrogen purge. The filtrate was dark yellow. The products were weighed and crushed in a mortar and pestle. The yield and mesotrione concentration of the respective complex is shown in Table 2.

TABLE 2
Urea complexes of mesotrione and a phosphate
ester surfactant by the evaporation method

| | | Mesotrione |
|---|---|---|
| Phosphate Ester used | Recovery | content |
| Klearfac ® AA 270 | 15.3 g | 6.6% |
| Lutensit ® AE-P | 14.3 g | 6.8% |
| Crodafos ™ SG | 14.0 g | 7.0% |
| Phospholan ™ PS-220 | 13.0 g | 5.7% |

### Example 3: Preparation of Urea Complexes of Tembotrione by Slow Cooling Method in an Oil Suspension

85.8 g of urea was combined with 24.2 g of water (corresponding to 110 g of 78% urea in water), and 4 g of Crodafos™ O10A in a 250-ml roundbottom flask. The flask was capped and placed in an oven held at 85° C. A bottle containing 2 g of Tween 61®, 3 g of Break-thru® EM O7 and 400 mL of isooctane was also placed in the same oven along with a 1-liter beaker. Once the reagents were hot, the flask was placed in an 85° C. oil bath and stirred. Once the urea was fully dissolved, 10.0 g of tembotrione was added to the mixture. As dissolution neared completion (about 3 minutes), the isooctane solution was poured into a beaker and mechanical stirring initiated in a hood without heating. The urea solution was then poured into the beaker and stirring continued for about five minutes as the emulsion slowly cooled. Precipitation was seen almost immediately (around 70° C.). The urea complex was recovered by filtration in a Buchner funnel, which was slowed by clogging of the frit. The urea complex recovered in the filter was dried overnight at 65° C. under 20″ Hg (about 0.67728 bar) vacuum with nitrogen purge, then crushed with a mortar and pestle. 85 g of the resulting material (from two batches) were combined with lignosulfonate dispersant Ultrazine™ NA. The product obtained had a tembotrione content of 8.63%.

### Example 4: Preparation of Urea Complexes of Atrazine by Hot Melt Extrusion

In this case, the complexes included atrazine, urea and solutions of Methocel® J12 MS. The water reduces the melting point of urea while the Methocel® serves as a binder. A waxy ethylene oxide-propylene oxide block copolymer, Pluronic® P123 (BASF) was included to provide lubrication in the extruder and to disperse the urea. An alcohol ethoxylate was included as non-ionic surfactant (constituent (c)) which also provides wetting. Some composition also used dispersants to promote the formation of fine atrazine particles when the complex was hydrolyzed. The alcohol ethoxylates used were Synperonic® A7 and Break-thru® EM O7. The dispersants were Agnique® DDL and Toximul® 8244. Compositions were prepared in a Haake™ Mini CTW hot melt extruder. This device is a small twin-screw extruder in which the components of the composition can be circulated prior to extrusion. All composition were prepared with a 95° C. extruder temperature and a screw speed of 90 rpm. With the extruder at temperature and the screws turning, Pluronic® P123 was added first for lubrication followed by the Methocel® solution and dispersants. The urea and atrazine were added together. The alcohol ethoxylate was added last. The mixture was then circulated in the extruder until molten and uniform, about two minutes. The compositions were easily crushed to a fine powder with a mortar and pestle. All dissolved very rapidly in water, forming 1-5 micron atrazine particles by optical microscopy. Settling was slow. The respective atrazine content of the compositions is shown in Table 3.

TABLE 3
Preparation of urea complexes of Atrazine by hot melt extrusion

| Ingredient | Complex 4-1 | | Complex 4-2 | | Complex 4-3 | |
|---|---|---|---|---|---|---|
| Atrazine | 3.4 | g | 3.4 | g | 3.4 | g |
| Urea | 0.65 | g | 0.65 | g | 0.65 | g |
| Methocel ® J12MS, | 0.2% solution of | | 0.4% solution of | | 1% solution of | |
| solution in water | Methocel ®, 0.6 g | | Methocel ®, 0.6 g | | Methocel ®, 0.6 g | |
| Pluronic ® P123 | 0.4 | g | 0.4 | g | 0.4 | g |

| Alcohol ethoxylate | Break-thru ® | Synperonic ® | | Synperonic ® | |
|---|---|---|---|---|---|
| | EM O7, 0.3 g | A7, 0.25 g | | A7, 0.25 g | |
| Agnique ® DDL | — | 0.15 | g | 0.15 | g |
| Toximul ® 8244 | — | 0.1 | g | 0.1 | g |

| | | | |
|---|---|---|---|
| % Atrazine | 14.0% | 12.5% | 12.6% |

### Example 5: Preparation of a Urea Complex of Tembotrione by Pouring a Fully Molten Mixture onto a Chilled Metal Plate

The composition also included a powder wetting agent, Agnique® ANS 3DNPW, a 2% solution of Methocel® A7 J12 MS in water, and an alcohol ethoxylate, Synperonic® 13/6. The composition was prepared on an 80 g scale, see Table 4 below. The urea, phosphate ester, wetting agent and Methocel® J12 MS solution were combined in a 250-mL roundbottom flask equipped with a stirring bar. The flask was capped and preheated in a 90° C. oven. The flask was transferred to a 105° C. oil bath and the tembotrione added and stirred until the solution was homogeneous. The Synperonic® 13/6 was added and the mixture poured in portions onto an aluminum plate sitting on ice. The thin layer of composition on the plate was scraped off and chopped into small pieces. Dissolution was easy and complete. A pool of three batches prepared in this way was analyzed as comprising 16.8 wt. % tembotrione.

TABLE 4
Preparation of a urea complex of Tembotrione by pouring
a fully molten mixture onto a chilled metal plate

| Ingredient | Wt. % | Amount | |
|---|---|---|---|
| Tembotrione | 19% | 15.2 | g |
| Urea | 61% | 48.8 | g |
| 2% Methocel ®J12MS in water | 8.5% | 6.8 | g |
| Synperonic ® 13/6 | 3.5% | 2.8 | g |
| Agnique ® PE TDA 9 | 5% | 4.0 | g |
| Agnique ® ANS 3DNPW | 3% | 2.4 | g |

When applied in a 2% solution of Sokalan® CP 9 this formulation provided effective fungal control for peanut plants in the field without phytotoxicity.

### Example 6: Preparation of a Urea Complex of Prothioconazole by Rapid Cooling of the Melt

The urea, Methocel® solution, and Agnique® ANS 3DNPW were combined in a 100 mL roundbottom flask equipped with a stirring bar that was placed in a 105° C. oven. See, Table 5 below. After transfer to a 105° C. oil bath, melting completed, and viscosity was low. The prothioconazole was added and given 10 minutes to fully dissolve before adding the alcohol ethoxylate. The composition was poured out in two operations onto the aluminum plate sitting on ice. Thin wafers were obtained. The wafers were broken into small pieces which dissolved rapidly in water, releasing very fine prothioconazole particles.

TABLE 5
Preparation of a urea complex of Prothioconazole
by rapid cooling of the melt

| Ingredient | Wt. % | Amount |
|---|---|---|
| Prothioconazole | 14% | 7.0 g |
| Urea | 72% | 36.0 g |
| Methocel ® J12MS, 2% in water | 8% | 4.0 g |
| Synperonic ® A7 | 3% | 1.5 g |
| Agnique ® ANS 3DNPW | 3% | 1.5 g |

### Example 7: Enhanced Herbicidal Efficacy of the Urea-Atrazine Complexes of Example 1

This was compared to conventional atrazine when used alone or in combination with the HPPD inhibitor herbicide mesotrione. 95 parts of the complex by weight (containing 9.9% atrazine and 10.7% total triazine compounds) was combined with 5 parts of the dispersant Morwet® EFW to provide a dispersible composition with a 9.4% atrazine loading. The composition, dispersed in water, was sprayed on velvetleaf (ABUTH) at rates of 140 and 280 g/ha and compared to a conventional commercial atrazine composition, AAtrex® Nine-O®, at the same rates. Tank mixtures with the mesotrione herbicide Callisto® (Syngenta) were also evaluated. The velvetleaf plants at the 6-9 leaf stage were sprayed with a 9501 flat-fan nozzle at 93 L/ha. Crop oil concentrate (COC) was included in some treatments at 1% vol/vol. For treatments which included Callisto®, the mesotrione rate was 17.5 g/ha at the lower atrazine rate (140 g/ha) and 35 g/ha at the higher atrazine rate (280 g/ha). Table 6 shows the % control of velvetleaf with conventional atrazine and urea-atrazine-complex.

TABLE 6
Control of velvetleaf with different atrazine containing materials

| | 140 g/ha atrazine | 280 g/ha atrazine |
|---|---|---|
| Treatment | (% control ABUTH) | (% control ABUTH) |
| AAtrex ® Nine-O ® + COC | 18.3 | 28.3 |
| Atrazine in urea, no COC | 30.8 | 40.8 |
| Callisto ® + AAtrex ® | 68.8 | 78.0 |
| Nine-O ® + COC | | |
| Callisto ® + atrazine- | 73.8 | 85.8 |
| in-urea + COC | | |
| Callisto ® + atrazine- | 56.3 | 68.3 |
| in-urea, no COC | | |

### Example 8: Enhanced Herbicidal Efficacy of the Urea-Tembotrione Complexes of Example 3

Control of velvetleaf (ABUTH) with the tembotrione complex was compared to a conventional tembotrione formulation, Laudis® without the use of adjuvants at a rate of 17.5 g/ha, i.e. well below the commercially recommended rate of 90 g/ha. Tank mixtures were sprayed on velvetleaf plants at 15 gallons per acre (approx. 140 L/ha). Plants were evaluated visually three weeks after spraying. At the time of evaluation, control was 13% with Laudis® and 22% with the urea complex composition.

### Example 9: Stability Studies of Urea Complexes of Mesotrione and Tembotrione of Examples 2 and 3 Respectively

Stability was assessed by placing the samples in an oven and sampling periodically. The mesotrione complexes were evaluated in a 54° C. oven over a 6-week period. All complexes exhibited acceptable stability, but the Phospholan™ PS-220 and Crodafos™ SG complexes performed particularly well.

Table 7 shows the concentration of mesotrione in the urea complexes of Example 2 after aging in a 54° C. oven.

TABLE 7
Stability study results for mesotrione concentration
in urea complex over 8 weeks at 54° C.

| | Concentration of mesotrione | | | | |
|---|---|---|---|---|---|
| Phosphate Ester | Week 0 | Week 1 | Week 2 | Week 4 | Week 6 |
| Klearfac ® AA 270 | 6.6% | 6.5% | 6.3% | 5.9% | 5.4% |
| Lutensit ® AE-P | 6.8% | 6.4% | 6.5% | 6.4% | 6.2% |
| TCrodafos ™ SG | 7.0% | 7.3% | 7.1% | 6.8% | 6.4% |
| Phospholan ™ PS-220 | 5.7% | 5.9% | 5.6% | 5.9% | 5.5% |

The tembotrione-urea complex of Example 3 was tested by aging in an oven at 54° C. for eight weeks. As shown in Example 3, inclusion of an acidic phosphate ester surfactant, Crodafos™ O10A, (PEG-10 oleyl phosphate, Croda) stabilizes tembotrione and mesotrione against chemical decomposition. Table 8 shows the tembotrione concentration in urea complex over 8 weeks at 54° C.

TABLE 8
Stability study results for tembotrione concentration
in urea complex over 8 weeks at 54° C.

| Week | % Tembotrione |
|---|---|
| 0 | 9.7 |
| 1 | 9.7 |
| 3 | 9.4 |
| 3 | 9.5 |
| 4 | 9.4 |
| 5 | 9.4 |
| 6 | 9.0 |
| 7 | 9.0 |
| 8 | 8.7 |

### Example 10: Treatment of Corn Seed with the Urea-Prothioconazole Complexes from Example 6

The urea composition was first added to water or a water-Sokalan® mixture in a plastic centrifuge tube and vortexed briefly to complete dissolution. Color and a standard seed treatment polymer were added to the tube. After brief vortexing, 8.6 mL of the mixture was applied to 1 kg of corn seed, corresponding to a rate of 12 fluid ounces per hundredweight seed. Treatment time was approximately 33 seconds.

Treatments were performed with rates of 0.021 and 0.085 mg prothioconazole per seed. Control compositions at both rates were performed with the prothioconazole suspension concentrate Proline® For the urea composition, equivalent amounts of water or water-Sokalan® were used. 20% Sokalan®-80% water was used at the 0.032 mg/seed rate and 30% Sokalan®-70% water (wt/wt) at the 0.085 mg/seed rate. The low-rate seed treatment solution prepared using Sokalan® was transparent prior to the addition of the seed colorant due to the small size of the prothioconazole particles formed.

Even at the high rate, there was no chalkiness due to urea on the surface and nothing transferred to a gloved hand when the seed was rubbed. At the high rate, the seed treated with the urea complex hydrolyzed in Sokalan® were noticeably glossier than control seeds or the seeds treated with the urea complex hydrolyzed in water alone.

### Example 11. Dynamic Light Scattering

### Example 11a: Dynamic Light Scattering of the Urea Complex of Tembotrione of Example 5

The tembotrione composition from Example 5, prepared by rapid cooling from the melt, was hydrolyzed and tembotrione particle evaluated by dynamic light scattering using a Malvern Nano-ZS Zetasizer. 0.6 g of the complex was added to 15 mL of water or 15 mL of 2% Sokalan® in a 30-mL vial and shaken to disperse. The solutions stood at room temperature for two hours prior to measuring particle size. The water hydrolysis was very slightly cloudy and the Sokalan® hydrolysis was optically clear. Dynamic light scattering measured a narrow particle size distribution for both hydrolysis mixtures with a mean particle size of 298 nm for the water hydrolysis and 5 nm for the Sokalan® hydrolysis. The remarkable Sokalan® hydrolysis result was replicated to ensure its accuracy.

### Example 11b: Dynamic Light Scattering of the Urea Complex of Prothioconazole from Example 6

Two batches were prepared which analyzed as 15.0% and 14.5% prothioconazole. When dispersed in water or 2% Sokalan® by the method described in Example 11, dynamic light scattering showed a sharp peak at 1.5 micron for Sokalan® dispersion and a broader peak between 1.2 micron and 2.1 micron for hydrolysis in water alone. The solution was analyzed with a Malvern Nano-ZS Zetasizer as in Example 11a.

### EXAMPLES A1-A9

### Example A1: Urea Complexes of Tetraniliprole Prepared by Hot Melt Extrusion without the Use of Water

This Example describes preparation of a urea complex of the insecticide tetraniliprole. The extrusion was performed at moderate temperature with high loading of the active ingredient and without the need to add water. This is an indication of a eutectic between tetraniliprole and urea. The composition is intended to be used in seed treatment, delivering tetraniliprole to the seed surface with low crystallinity in order to enhance bioavailability. Remarkably, when this composition is added to water, tetraniliprole is released as a liquid in the form of a stable emulsion, which is ideal for the intended application.

Ten batches of 5 g each of the following product were prepared by extrusion of the mixture with the Haake™ Mini CTW extruder described in Example 4. Extrusion was performed at 105° C. with a screw speed of 90 rpm. The composition extruded easily as a continuous “noodle” and was easily crushed to a powder after cooling to room temperature.

TABLE A1
Composition of urea complexes of tetraniliprole prepared
by hot melt extrusion without the use of water

| | |
|---|---|
| Tetraniliprole | 28% |
| Agnique ® ANS 3DNPW | 4% |
| Synperonic ® 13/6 | 3% |
| Toximul ® 8244 | 3% |
| Urea prills | 62% |

After crushing, the composition dissolved almost instantly in water to form a bright white emulsion of liquid tetraniliprole in water. No crystallization or loss of emulsion quality was noticeable after standing overnight at room temperature.

### Example A2: Treatment of Corn Seed with Tetraniliprole in Urea

The ten batches of the tetraniliprole in urea composition from Example A1 were pooled and a portion used to treat corn seeds. 1 kg of corn seed with a BT trait (Bacillus thuringiensis proteins which protect the plant from insects) was treated with a ratio of treatment solution to seed mass of 16 fluid ounces per hundredweight corn seed. Corn seed, 1600 seeds per pound, was treated in a bowl treater with a solution comprising the urea complex of Example A1 at rates of 0.125 and 0.25 mg tetraniliprole per seed. The treatment mixture also comprised the fungicide fluoxastrobin at 0.085 mg/seed as well as color and a binder polymer. In order to optimize the tetraniliprole emulsion, the treatment solution further comprised Soprophor® FLK at a rate of 0.6 fluid ounces per hundredweight.

The treated seed was found to have equivalent warm and cold germination to untreated seed. However, it was unsuitable for efficacy testing because of the BT trait. Therefore, the treatments repeated on a smaller scale with corn seed that did not contain a BT trait at rates of 0.063 and 0.125 mg tetraniliprole per seed. Treatments were also performed with a conventional suspension concentrate (“SC”, 480 g/L) of tetraniliprole. These batches were used in the efficacy assay described in Example A3.

### Example A3: Efficacy of a Tetraniliprole-In-Urea Complex as a Seed-Applied Insecticide

The non-BT seed treated at 0.063 mg/seed as described in Example A2 germinated and developed in pots. When the plants were about a foot high, a black cutworm was introduced and damage to the stalk was measured. At the low rate of tetraniliprole, the urea complex applied with Soprophor® FLK provided better protection to the stalk than the conventional suspension concentrate as shown in Table A3-1.

TABLE A3-1
Black cutworm stalk damage to corn seedlings
treated with 0.063 mg//seed tetraniliprole

| Treatment | Stalk damage (%) |
|---|---|
| Urea complex + Soprophor ® FLK | 31% |
| Conventional suspension concentrate | 43% |
| Untreated | 80% |

The non-BT seed treated at 0.125 mg/seed as described in Example A2 were evaluated based on reduction of the area of the third leaf due to black cutworm feeding. As shown in the Table A3-2, the urea complex again out-performed a conventional suspension concentrate.

TABLE A3-2
Reduction in third leaf surface area for corn seedlings treated
with 0.125 mg/seed tetraniliprole after black cutworm feeding

| | 3^{rd} leaf surface |
|---|---|
| Treatment | area reduction (%) |
| Urea complex + Soprophor ® FLK | 45% |
| Conventional suspension concentrate | 60% |
| Untreated | 96% |

### Example A4: Tembotrione-In-Urea Compositions Incorporating Different Surfactants which Influence the Form of the Active Ingredient after Hydrolysis

This Example describes the preparation of two tembotrione complexes by hot melt by pouring the melt onto a cold metal plate. In addition, the two compositions incorporate different surfactants which induce different behavior when the respective composition is added to water. One composition, A4-1, forms nanoparticles similar to the Example 5 complex whose particle size was characterized in Example 11. The other composition A4-2 forms an emulsion of liquid tembotrione. Also, these compositions illustrate the incorporation of methanesulfonic acid (Lutropur® MSA was used) to reduce the pH and provide chemical stability. As in Example 5, incorporation of an un-neutralized phosphate ester, Agnique® PE TDA 9, provided additional acidity.

Complex A4-1, which forms nanoparticles, was prepared from the melt at 100 g scale similar to Example 5. The Geropon, Agnique® PE TDA 9, Synperonic® 13/6, water, and urea were combined in a 250-mL flask equipped with a stirring bar, capped, and held in an oven at 105° C. overnight. The flask was transferred to a 105° C. oil bath. 0.5 g of MSA was added followed by tembotrione and stirring maintained until the tembotrione dissolved and a good vortex established. The mixture was poured out in batches forming a sheet of soft material with a red color. The product was dried for 4 hours at 55° C. under 24″ Hg (about 0.81273 bar) vacuum with nitrogen purge.

Complex A4-2 a scale of about 5 g in the Haake™ CTW hot melt extruder. The components added to the extruder are shown in the table. Multiple batches were prepared at an extruder temperature of 90° C. and a screw speed on 90 rpm. The batches were pooled to enable efficacy testing.

TABLE A4
Tembotrione compositions containing methanesulfonic acid

| Ingredient | Complex A4-1 | Complex A4-2 |
|---|---|---|
| Tembotrione, technical grade | 21% | 21% |
| Urea prills | 61% | 63% |
| Lutropur ® MSA | 1% | 1% |
| Agnique ® PE TDA 9 | 3% | 4% |
| Synperonic ® 13/6 | 4% | — |
| Geropon ® TA/72 | 4% | — |
| Deionized water | 6% | — |
| Makon ® TSP-60 | — | 11% |
| Tembotrione wt. % loading | 18.7% | 18.8% |

For tembotrione, incorporation of a polymeric dispersant which stabilizes solid particle surfaces leads to nanoparticle formation with composition A4-1. Geropon® TA/72, a polymeric dispersant from Solvay was used for this purpose.

In contrast, composition A4-2 includes Makon® TSP-60, which promotes the formation of emulsions, leading to the formation of an emulsion of liquid tembotrione when complex A4-2 is added to water.

### Example A5: High-Loading Fluopyram-In-Urea Complexes by Hot Melt Extrusion

This Example illustrates the high loading that can be achieved in urea complexes of actives with melting points near the extrusion temperature, which favors the formation of eutectics. It also shows that release of the active as a liquid when the complex is hydrolyzed is easily achieved while varying the emulsifier and alcohol ethoxylate in the composition.

Fluopyram is a fungicide with a melting point of 118° C., having poor water solubility of 16 mg/L, but highly soluble in polar organic solvents (>250 g/L in ethyl acetate or DMSO). Approximately 30% (nominal) compositions of fluopyram in urea were prepared using the Haake™ Mini CTW hot melt extruder at 90° C. with a screw speed of 90 rpm. In all cases extrusion was easy, producing a continuous “noodle” which crushed easily after cooling. The compositions were prepared at approximately 5 g scale.

1.5 g of fluopyram, 2.7 g of urea prills, and variable quantities of alcohol ethoxylate, wetting agent, and emulsifier were added to the Haake™ Mini CTW, circulated until homogeneous, and then extruded at the conditions indicated above.

The following three composition A5-1, A5-2 or A5-3 were prepared:

### Composition A5-1:

1.5 g of fluopyram+2.7 g of urea prills+0.4 g Makon® TSP-60+0.4 g Synperonic® 13/6

### Composition A5-2:

1.5 g of fluopyram+2.7 g of urea prills+0.4 g Makon® TSP-60+0.4 g Break-thru® EM O7

### Composition A5-3:

1.5 g of fluopyram+2.7 g of urea prills+Break-thru® DA-647+0.3 g Myrj S8+Agnique® ANS 3DNPW

All compositions produced an emulsion of liquid fluopyram when added to water in a ratio by weight of about 10:1 of water to Composition A5-1, A5-2 or A5-3, respectively. The emulsions were stable for about two hours at which point some crystallization and settling occurred. The fluopyram particles formed in this process re-dispersed easily and did not appear crystalline by optical microscopy.

### Example A6: Atrazine-In-Urea Complex Prepared from the Melt

This Example describes preparation of an atrazine-in-urea complex from the melt in contrast to the evaporation method or hot melt extrusion in Example 1 and Example 4 respectively. This composition also proved to have significantly better pre-emergent efficacy than a conventional suspension concentrate. On a 150 g scale, an atrazine-in-urea complex was formed with the components shown in Table A6.

TABLE A6
Atrazine-in-urea complex obtained from the melt

| Ingredient | Wt. % | Amount | |
|---|---|---|---|
| Atrazine | 16% | 24 | g |
| Urea | 68% | 102 | g |
| Methocel ® J12MS, 2% solution in water | 6% | 9 | g |
| Synperonic ® A7 | 5% | 7.5 | g |
| Break-thru ® S233 | 5% | 7.5 | g |

All of the components except the atrazine and 3 mL of the Methocel® J12 MS solution were combined in a 500-mL roundbottom flask equipped with a stirring bar. The flask was capped and placed in an oven at 115° C. overnight. The flask was transferred to a 120° C. oil bath and stirred. The remaining Methocel® J12 MS solution was added, and the atrazine added in portions while maintain stirring. When dissolution was complete, the resulting mixture was poured out onto an aluminum plate sitting on a bed of ice. In two pours, 130.9 g of a white composition was recovered. The batch was annealed overnight under nitrogen at 70° C. which reduced the mass to 123.9 g. The annealed composition was crushed to a fine powder with mortar and pestle. The resulting product had an atrazine content of 17% due to evaporation of water.

### Example A7: Pre-Emergent Efficacy of Urea Complexes of Atrazine and Tembotrione Against Velvetleaf

This Example demonstrates that the atrazine-in-urea complex from Example A6 alone or in combination with tembotrione-in-urea from Example 5 exhibits superior pre-emergent control of the large-seeded broadleaf weed velvetleaf (ABUTH) compared to conventional commercial suspension concentrate compositions of these actives. In this experiment, as comparative compositions the commercially available products AAtrex® Nine-O® for atrazine and Laudis® for tembotrione were used.

Spray solutions of the compositions were applied to soil at a rate of 15 gallons per acre (approx. 140 L/ha). The rates for each herbicide are shown in Table A7. For each treatment six velvetleaf seeds were placed ½-inch below the soil surface and given two weeks to emerge. For each seed, the ratio of emerged velvetleaf fresh weight to the average fresh weight of six velvetleaf seeds that were not treated with herbicide was determined. Percent control, shown in Table A7, was calculated from these values where 100% control would correspond to zero fresh weight of emerged velvetleaf.

The efficacy results show that the atrazine composition from Example A6 is far superior to conventional atrazine for control of velvetleaf. Typically, large-seeded broadleaf weeds such as velvetleaf present the greatest challenge for pre-emergent control and provide a clear assessment of relative efficacy.

TABLE A7
Control of velvet leaf using atrazine, as single active
ingredient or in combination with tembotrione

| | | Atrazine rate | Tembotrione | % |
|---|---|---|---|---|
| | Tembotrione | (active | rate active | control |
| Atrazine material | material | ingredient) | ingredient) | ABUTH |
| AAtrex ® Nine-O ® | — | 140 g/ha | — | 22.5% |
| Atrazine-urea, | — | 140 g/ha | — | 70.1% |
| from Example A6 | | | | |
| AAtrex ® Nine-O ® | Laudis ® | 140 g/ha | 23 g/ha | 55.8% |
| Atrazine-urea, | Tembotrione-urea, | 140 g/ha | 23 g/ha | 80.7% |
| from Example A6 | from Example 5 | | | |

### Example A8: Efficacy of Tembotrione Released from Compositions According to the Invention in Liquid Form or as Nanoparticles

This Example demonstrates that urea complexes which release the active as nanoparticles or liquids when hydrolyzed can exhibit improved foliar efficacy compared to conventional suspension concentrate compositions which are suspensions of small solid particles of the active. This is illustrated with the tembotrione-in-urea compositions from Example A4 above.

The post-emergent efficacy of the tembotrione-in-urea compositions from Example A4 was evaluated in a greenhouse trial. Compositions A4-1 (releasing tembotrione nanoparticles) and A4-2 (releasing tembotrione in liquid form as an emulsion) were applied to Palmer amaranth (AMAPA) and veltvetleaf (ABUTH) plants at ⅓ and ½ of the usual rate, i.e. 30 g/ha and 46 g/ha of tembotrione. The compositions, along with a commercial tembotrione suspension concentrate, Laudis® (Bayer) were applied to 4″-6″ (10.16-15.24 cm) plants. All applications were performed with and XR9501R nozzle (Teejet) at a spray rate of 15 gallons per acre (approx. 140 L/ha). Plant injury was evaluated 21 days after treatment. Six plants were used for each treatment.

Average injury is shown in the Table A8-1 below. Both compositions A4-1 and A4-2 provided superior control of both weeds compared to a conventional suspension concentrate composition. In most cases, tembotrione released in liquid form resulting from composition A4-2 was more efficacious than the tembotrione in nanoparticle form resulting from composition A4-1.

The efficacy of three different tembotrione compositions was assessed: For the commercial product Laudis®, composition A4-1 (producing tembotrione nanoparticles) and composition A4-2 (producing liquid tembotrione) against Palmer amaranth (AMAPA) and velvetleaf (ABUTH) at 30 g/ha and 46 g/ha of tembotrione. The level of control (injury) was assessed in percent 21 days after treatment (21 DAA).

TABLE A8-1
Control (Injury) 21 DAA of Palmer amaranth (AMAPA)
and velvetleaf (ABUTH) of different tembotrione
compositions at dose rates 30 g/ha and 46 g/ha

| | AMAPA | AMAPA | ABUTH | ABUTH |
|---|---|---|---|---|
| | Injury, | Injury, | Injury, | Injury, |
| Composition | 30 g/ha | 46 g/ha | 30 g/ha | 46 g/ha |
| Laudis ® | 35% | 57% | 50% | 55% |
| A4-1 (Example A4) | 41% | 63% | 60% | 65% |
| A4-2 (Example A4) | 69% | 80% | 58% | 88% |

Tembotrione is typically used with an adjuvant. The three compositions were also evaluated with the adjuvant AU-973 (Adjuvants Unlimited, Memphis TN) at 0.25% vol/vol in the application spray mixture using the same protocol at a tembotrione rate of 30 g/ha.

The efficacy of three different tembotrione compositions was assessed” For the commercial product Laudis®, and compositions A4-1 (producing tembotrione nanoparticles) and A4-2 (producing liquid tembotrione) against Palmer amaranth (AMAPA) and velvetleaf (ABUTH) at 30 g/ha with the adjuvant AU-973 included in the application spray mixture at 0.25% vol/vol. The level of control (injury) was assessed in percent 21 days after treatment.

TABLE A8-2
Control (Injury) 21 DAA of Palmer amaranth (AMAPA) and
velvetleaf (ABUTH) of different tembotrione compositions
at a dose rate of 30 g/ha with additional adjuvant

| | AMAPA | ABUTH |
|---|---|---|
| | Injury, | Injury, |
| Composition | 30 g/ha | 30 g/ha |
| Laudis ® | 55% | 55% |
| A4-1 (Example A4) | 68% | 87% |
| A4-2 (Example A4) | 91% | 92% |

Comparing the results, shown in the Table 8-2, to those at 30 g/ha in Table 8-1 shows that the adjuvant improved weed control for both weeds and all compositions. When using the adjuvant, once again, both composition A4-1 and A4-2 were superior to the conventional composition, with composition A4-2 performing as the best of all.

### Example A9: Differential Scanning Calorimetry (DSC) of a Tetraniliprole-Urea Composition

The tetraniliprole-urea composition of Example A1 was modified by addition of a further non-ionic surfactant as emulsifier, Makon® TSP-60, in the composition. An additional surfactant, sodium lauryl sulfate (Stepwet® DF-95) was also included in the composition to further aid in emulsification. This anionic surfactant likely is not to be part of the urea-tetraniliprole phase.

This composition-shown in Table A9-produced an emulsion of liquid tetraniliprole that was stable overnight (approx. 16 h) at ambient temperature (approx. 20° C.) when the composition was hydrolyzed in 8 times the amount by weight of water without the need to add an additional emulsifier. The composition of Table A9 was prepared by hot melt extrusion using the same procedure as in Example A1.

TABLE A9
Tetraniliprole-urea composition prepared by hot melt
extrusion which includes an emulsifier, Makon ® TSP-
60, and anionic surfactant Stepwet ® DF-95.

| | |
|---|---|
| Tetraniliprole | 26% |
| Makon ® TSP-60 | 9% |
| Agnique ® ANS 3DNPW | 4% |
| Synperonic ® 13/6 | 3% |
| Stepwet ® DF-95 | 7% |
| Urea prills | 51% |

Differential scanning calorimetry was performed on this composition and on the urea prills used to prepare it. Urea prills exhibit a simple melting peak at 134° C. The DSC data of the tetraniliprole-urea composition in this Example also exhibits a single melting peak, but at a much lower temperature, at about 125° C.

The absence of a melting peak at 134° C. demonstrates that the product is not a mechanical mixture of urea starting material (urea prills) and the active ingredient tetraniliprole. Instead, a modified urea matrix structure incorporating the active ingredient and some surfactant constitutes the only detectable phase. The reduced melting point reflects disruption to the structure of urea due to incorporation of the other components.

These DSC data cannot exclude the possibility that a minor amount of tetraniliprole was still present as a separate phase (the tetraniliprole melting point, 228° C., was outside of the DSC measurement range). However, the DSC data of urea prills and the tetraniliprole-in-urea composition of Example A9 showed the complete disappearance of the urea phase from the starting material and the formation of a lower-melting phase comprising tetraniliprole as a dispersion in urea.

### EXAMPLES B10-B19 AND COMPARATIVE EXAMPLE

### Example B10

This Example describes a high-loading (32.6% phenmedipham) urea-complex of the herbicide phenmedipham which readily hydrolyzes to a fine emulsion. The composition was prepared on a 4.6 g scale by feeding a solid premix which had been ground in a knife mill to the using the Haake™ Mini CTW extruder at 80° C. with a screw speed of 90 rpm. The premix consisted of 1.5 g of phenmedipham technical, 0.5 g of Makon® TD-12, and 2.6 g of urea. The knife mill processing served to disperse the liquid surfactant over the solid components of the composition. This composition circulated well in the extruder and extruded as a good though fragile “noodle” that was easily crushed to a powder.

### Example B11

This Example illustrates the process by which a composition equivalent to that of Example B10 can be prepared on a 100 g or larger scale in a mixer. In addition, the scaled-up process enables “flood feeding” of the raw material. This means that the feed, a powder, flows freely and can be taken up by the screws of the mixers without manual intervention. Such a process is readily scalable to larger equipment while being convenient at the scale shown in this example. A Readco Kurimoto RK1 twin screw mixer was employed. While more powerful with significantly greater throughput, the Readco Kurimoto RK1 had a different design than the Haake™ Mini CTW extruder used in the preceding Examples.

To a 250 mL Waring spice grinder bladed blending cup was added about 56 g of urea prills, followed by about 33 g of phenmedipham technical powder, and then about 11 g of molten Makon® TD-12 held at 60° C. This was blended in the spice grinder with frequent pulsing and breaks for scraping down the sides of the container, which quickly became sticky. A lightly prilled yet soft material was poured out, then the remainder scraped off the sides and re-blended before pouring out. This process was repeated for a total of 200 g of material. This feed was used for a 100 g scale preparation of the composition on the Readco Kurimoto RK-1 mixer with the following settings.

| | |
|---|---|
| Amount | 100 g |
| Waste gate | 20% open (4 mm) |
| Screw speed | 30 rpm |
| Feed | Flood |
| Barrel set pt. | 200° F. (about 93.3° C.) |
| Barrel temp | 200° F. (about 93.3° C.) |
| Product temp | 205° F. (about 96.1° C.) |

The sample was added to the hopper in one portion once the barrel was at temperature, flood feeding the screws. The motor was then started, letting the screws turn. The material extruded at a moderate pace, leaving a friable, shark-skinned, thick flat (4 mm×25 mm) “noodle”. The product was off-white and of good quality, with adequate strength and brittleness for crushing. Grinding yielded a very fine powder which dispersed rapidly in water.

### Example B12

This Example describes a tembotrione-urea composition with excellent dispersion in water and easy scaleup from the small extruder to production with a continuous mixer. The tembotrione technical used had a purity of 96%. The composition was as follows:

| | |
|---|---|
| Tembotrione technical | 27% |
| Stepfac ® 8181 | 4% |
| Makon ® TSP-12 | 6% |
| Toximul ® 8320 | 1% |
| 85% H₃PO₄ | 1% |
| Urea | 61% |

The composition was prepared on a 5 g scale with the Haake™ Mini CTW extruder operating at 80° C. with 90 rpm screw speed. A blend of the materials which had been ground in a small knife mill could be easily flood-fed to the extruder, resulting in a near-ideal peak torque of 1.2 N-m. The product extruded as a soft “noodle” which hardened upon cooling.

The same composition was then prepared on a 125 g scale on the Readco Kurimoto RK-1 mixer operating at 81° C. with a screw speed of 90 rpm. After grinding the materials in a knife mill, the composition was flood-fed to the Readco Kurimoto RK-1 inlet, emerging in about a minute as soft mass that was allowed to cool and harden. Power demand was low (5.7 A) due to the favorable flow properties of this composition. The product was ground to a powder in a Waring blender.

### Example B13

This Example describes the preparation of a high-loading urea complex of the herbicide safener cyprosulfamide. A premix of the following composition was ground briefly in a Waring spice grinder. 5 g of the premix were fed to a Haake™ Mini CTW twin-screw extruder operating at 80° C. with a screw speed of 90 rpm.

| | |
|---|---|
| Cyprosulfamide technical | 30% |
| Makon ® TSP-12 | 10% |
| Urea (prills) | 60% |

The extruded product underwent efficient dispersion of cyprosulfamide when added to water.

### Example B14

This Example describes the dispersion of active ingredients when compositions of the present invention are hydrolyzed. 0.2 g of the formulation was added to 10 mL of water in a vial and vortexed briefly. Urea dissolved, resulting in a dispersion of the active ingredient in water. The particle size distribution was measured immediately thereafter using a Malvern Mastersizer.

The particle size distribution measured for the phenmedipham composition of Example B11 and the cyprosulfamide composition of Example B13. Almost all of the respective active ingredient was present as a fine emulsion with a volume-weighted mean diameter (VMD or Dv50) of 1.2 micron for phenmedipham and 0.8 micron for cyprosulfamide. A broad shoulder at higher diameter corresponds to a small fraction of the formulation which had not completed dissolution.

The narrow distribution indicates that the active ingredient is dispersed as an emulsion rather than suspended solid both because the 1 micron size is characteristic of oil-in-water emulsions such as milk and because the narrow size range represents an equilibration as compared to the distribution of particle sizes which is expected from irreversible crystallization in a dispersion of solids.

### Example B15

This Example describes the preparation of a formulation of the fungicide prothioconazole at larger scale and its ability to form either a stable dispersion or a nanodispersion. It also illustrates the use of a second phase comprising anionic dispersants to accelerate the dispersion of the formulation in water. In this example, two anionic dispersants were used: Dowfax® 3B2 and Sokalan® CP-9.

Initial mixing of the following composition was performed in a Hobart planetary mixer at 500 g scale by first adding urea prills and dry Sokalan® CP-9 dispersant followed by the liquid ingredients. The prothioconazole technical powder was added last. After mixing, the resulting mixture appeared to be swollen urea prills.

| | |
|---|---|
| Prothioconazole technical | 28% |
| Dowfax ® 3B2 | 6% |
| Sokalan ® CP-9 (solid) | 4% |
| Toximul ® 8320 | 1% |
| Urea | 61% |

The mixture was then processed on a Readco Kurimoto RK-1 twin-screw mixer at 74° C. (165° F.) with a screw speed of 60 rpm. After cooling, the hard white product was ground with a Waring spice grinder and passed through a 850 micron sieve. The product dissolved instantly in water to form a milky dispersion which was stable overnight. When the product was dissolved in a 0.5% solution of the anionic Sokalan® CP-9 (solids basis), a clear solution was obtained due to the formation of prothioconazole nanoparticles. The suspension remained clear for more than a week.

### Example B16

This Example describes the preparation of a formulation at larger scale using an anionic dispersant to improve dispersion of the product when added to water. A formulation of the herbicide isoxaflutole was prepared with the following composition. The isoxaflutole technical used had a purity of 99% resulting in the formulation having an overall loading of about 32.7% isoxaflutole.

| | |
|---|---|
| Isoxaflutole technical | 33% |
| Crodafos ® T6A | 3% |
| 85% phosphoric acid | 1% |
| Dowfax ® 8390 | 3% |
| Soprophor ® CY/8 | 3% |
| Urea | 57% |

The urea prills were added to the bowl of a planetary mixer (Kitchen Aid) and a mixture of the liquid components (all materials except isoxaflutole) mixed in over about three minutes with continuous low agitation. The isoxaflutole technical was then added over about five minutes as agitation continued. The urea prills appeared to be noticeably swollen.

The product from the planetary mixer was gravity-fed to a Readco Kurimoto RK-1 twin-screw mixer at 75° C. (167° F.) with a screw speed of 60 rpm. A thin fragile “noodle” was produced which crushed easily and exhibited fast dissolution in water.

### Example B17

This Example demonstrates the superior post-emergent efficacy of the tembotrione-urea composition A4-2 from Example A4-releasing tembotrione in liquid form as an emulsion-against two difficult-to-control grasses: barnyardgrass (ECHCG) and crabgrass (DIGSA). Efficacy was evaluated at ⅓ and ½ of the usual rate, i.e. 30 g/ha and 46 g/ha of tembotrione and compared to the commercial tembotrione suspension concentrate Laudis® (Bayer). Application was performed without adjuvant at both rates and in addition, efficacy was assessed at 30 g/ha tembotrione with the addition of 0.25% vol/vol AU-973 (Adjuvants Unlimited, Memphis, TN) nonionic surfactant in the spray mixture. All applications were performed with and XR9501R nozzle (Teejet) at a spray rate of 15 gallons per acre (approx. 140 L/ha). The level of control of the respective weed was assessed in percent 21 days after treatment (21 DAA) in percent injury.

As shown in Table B17, the conventional formulation was ineffective on both grasses tested at both rates while high efficacy was seen for the composition A4-2 from Example A4, particularly for barnyardgrass (ECHCG).

TABLE B17
Control (Injury) of barnyardgrass (ECHCG) and crabgrass
(DIGSA with different tembotrione compositions at
dose rates 30 g/ha and 46 g/ha assessed 21 DAA

| | ECHCG | ECHCG | DIGSA | DIGSA |
|---|---|---|---|---|
| | Injury, | Injury, | Injury, | Injury, |
| Composition | 30 g/ha | 46 g/ha | 30 g/ha | 46 g/ha |
| Laudis ® alone | 0% | 0% | 0% | 0% |
| Laudis ® + AU 973 | 89.2% | | 90.8% | |
| A4-2 (Example A4) alone | 92% | 97.5% | 45.0% | 44.2% |
| A4-2 (Example A4) + AU-973 | 99.5% | | 91.7% | |

### Example B18

Example 9 demonstrated that pH-sensitive active ingredients such as mesotrione and tembotrione can be stabilized by inclusion in urea along with non-ionic surfactants such as phosphate ester surfactants in acid form. This Example B18 shows that stabilization can also be achieved by including phosphoric acid to the composition, with or without the presence of phosphate esters. It also demonstrates improved, i.e. higher, loading of tembotrione.

Four compositions of tembotrione in urea were prepared on a 5 g scale each using the Haake™ Mini CTW extruder operated at 80° C. with a 90 rpm screw speed. The tembotrione technical had a purity of 96%. A tristyrylphenol ethoxylate (Soprophor® S-25/80) and phosphoric acid were used to stabilize the tembotrione. The compositions also included a linear nonionic surfactant, either a phosphate ester or a linear alcohol ethoxylate.

0.3 g of the respective linear nonionic surfactant was added to the extruder first. The remaining 4.7 g had been pre-mixed and processed in a small knife mill prior to addition to the extruder. All four compositions extruded as good “noodles” which were crushed to powders prior to conducting the thermal stability trial.

### Tembotrione Compositions of Example B18:

| | |
|---|---|
| Tembotrione technical | 26% |
| Soprophor ® S-25/80 | 8% |
| Linear surfactant | 6% |
| Phosphoric acid (85% in water) | 1% |
| Urea prills | 59% |

The products, in powder form, were stored at 54° C. in an oven for four weeks. Samples were taken periodically and analyzed for their tembotrione content. Because of the small scale of preparation there were small differences in the initial tembotrione concentration, but all compositions exhibited excellent high-temperature stability of tembotrione.

TABLE B18
Tembotrione content (wt. %) of Example
B18 compositions - storage at 54° C.

| Linear surfactant | Week 0 | Week 1 | Week 2 | Week 4 |
|---|---|---|---|---|
| Agnique ® PE TDA 9 | 26.2% | 25.2% | 25.1% | 24.8% |
| Crodafos ® SG | 24.8% | 24.2% | 25.0% | 24.0% |
| Crodafos ® T6A | 26.8% | 28.9% | 27.0% | 27.2% |
| Makon ® TD-12 | 24.9% | 24.6% | 24.4% | 24.9% |

Example B19: The prothioconazole-urea compositions of Example B15 were analyzed by Differential Scanning Calorimetry (DSC), both after blending in a Hobart mixer and after subsequent hot blending in a Readco Kurimoto RK-1 twin screw mixer. The DSC data of both prothioconazole-urea compositions showed no trace of the crystalline prothioconazole peak, i.e. the crystalline prothioconazole used as starting material for producing the prothioconazole-urea compositions of Example B15 was not present anymore. These DSC data evidence that crystalline prothioconazole was absent in these prothioconazole-urea compositions and that a solid solution of prothioconazole in urea had formed. These DSC data also showed that the urea peak had been replaced by a broader peak at a lower temperature.

### COMPARATIVE EXAMPLE

This Example demonstrates that the compositions of the present invention are not obtained by precipitation from methanol with oleic acid.

This comparative example was conducted using the method of precipitation in methanol with oleic acid reported by Thakral, S.; Madan, A. K. for example in J. Pharmacy Pharmacology, 2007, 59, 1501-7, or in J. Incl. Phenom. Macrocycl. Chem., 2008, 60, 203-9 or in J. Pharm, Innov., 2008, 3, 249-57. The description of the procedure used in these publications is largely identical.

In these procedures, a solution of 0.5 g of the respective active ingredient (enalapril maleate, glipizide, and nicorandil) and 5 g of urea in 30 mL of methanol was prepared “by slight heating.” Subsequently, 0.6 g of oleic acid was added which “led to immediate precipitation.” After standing for 2-3 hours, the precipitate was collected by vacuum filtration.

This procedure was adapted to attempt to analogously obtain a tembotrione-urea complex.

A solution of 0.5 g of tembotrione and 5 g of urea in 50 mL of methanol was prepared by gentle heating. Subsequently, 0.6 g of oleic acid were added, and heating was discontinued. No precipitation occurred either at the time of addition or after allowing the mixture to cool to room temperature.

In contrast to the methods and procedures described in the context of the present invention, no single-phase tembotrione-urea complexes was obtained following the procedure of precipitation in methanol with oleic acid.

### EMBODIMENTS

For further illustration, additional non-limiting embodiments of the present invention are set forth below.

Embodiment 1. A composition in solid form at 25° C. and 1013 mbar comprising:
(a) a total amount of at least about 5% by weight of one or more pesticidal or pharmaceutical active ingredients having a melting point of at least 55° C. at 1013 mbar and a solubility of 50 g/L or less in deionized water having a pH of about 7 at 25° C. and 1013 mbar, constituent (a) not being present in form of a salt with an inorganic counter-ion (i.e. not as salt with an inorganic counter-ion such as monovalent metal ions, divalent metal ions, trivalent metal ions or an ammonium counter-ion),
(b) a total amount of at least about 50% by weight of urea,
(c) a total amount of at least about 1% by weight of one or more non-ionic surfactants,
   wherein the ratio by weight of the total amount of constituent (c) to the total amount of constituent (a) is about 0.8 or lower, and
   wherein the amounts indicated in each case are based on the total weight of the composition.

Embodiment 2. The composition of Embodiment 1, comprising
constituent (a) in a total amount of 5% or more by weight, of 6% or more by weight, of 7% or more by weight, preferably of 8% or more by weight, more preferably of 10% or more by weight, typically in the range of from 5% to 35%, preferably in the range of from 6% to 33%, preferably in the range of from 7% to 32%, preferably in the range of from 8% to 31%, more preferably in the range of from 10% to 30%,
constituent (b) in a total amount of 50% or more by weight, of 60% or more by weight, typically in the range of from 50 to 90% by weight, preferably in the range of from 60% to 85% by weight, often in the range of from 65% to 80% by weight, and
constituent (c) in a total amount of 2% or more by weight, 3% or more by weight, 4% or more by weight, preferably of 5% or more by weight, more preferably of 8% or more by weight, often of 10% or more by weight.

Embodiment 3. The composition of Embodiment 1, comprising
constituent (a) in a total amount of 7% or more by weight, preferably of 8% or more by weight, more preferably of 10% or more by weight,
and/or
constituent (c) in a total amount of 2% or more by weight, or of 3% or more by weight,
   wherein the amounts indicated in each case are based on the total weight of the composition.

Embodiment 4. The composition of Embodiment 1 or 2, comprising
constituent (a) in a total amount in the range of from 5% to 35% by weight, preferably in the range of from 6% to 33% by weight,
constituent (b) in a total amount in the range of from 50 to 90% by weight, preferably in the range of from 60% to 85% by weight,
and
constituent (c) in a total amount of 3% or more by weight, preferably of 4% or more by weight,
   wherein the amounts indicated in each case are based on the total weight of the composition.

Embodiment 5. The composition of any one of Embodiments 1 to 4, comprising
constituent (a) in a total amount in the range of from 8% to 35% by weight or in the range of from 10% to 33% by weight,
and/or
constituent (b) in a total amount in the range of from 55 to 85% by weight or in the range of from 60% to 85% by weight,
   wherein the amounts indicated in each case are based on the total weight of the composition.

Embodiment 6. The composition of any one of Embodiments 1 to 5, wherein the melting point of constituent (a) is in the range of from about 55° C. to about 350° C. at 1013 mbar.

Embodiment 7. The composition of any one of Embodiments 1 to 5, wherein the melting point of constituent (a) is in the range of from about 60° C. to about 300° C. at 1013 mbar.

Embodiment 8. The composition of any one of Embodiments 1 to 7, wherein the one or more active ingredients of constituent (a) have a molecular weight of less than 800 Dalton, preferably in the range of from 200 to 800 Dalton, preferably in the range of from 200 to 700 Dalton, more preferably in the range of from 210 to 600 Dalton, often in the range of from 300 to 600 Dalton or in the range of from 320 to 500 Dalton.

Embodiment 9. The composition of any one of Embodiments 1 to 8, wherein the ratio by weight of the total amount of constituent (c) to the total amount of constituent (a) is in the range of from about 0.1 to about 0.65, based on the total weight of the composition.

Embodiment 10. The composition of any one of Embodiments 1 to 8, wherein the ratio by weight of the total amount of constituent (c) to the total amount of constituent (a) is in the range of from about 0.2 to about 0.55, based on the total weight of the composition.

Embodiment 11. The composition of any one of Embodiments 1 to 8, wherein the ratio by weight of the total amount of constituent (c) to the total amount of constituent (a) is in the range of from about 0.25 to about 0.5, based on the total weight of the composition.

Embodiment 12. The composition of any one of Embodiments 1 to 11, wherein the one or more active ingredients of constituent (a) have a solubility of 20 g/L or less in deionized water having a pH of about 7 at 25° C. and 1013 mbar.

Embodiment 13. The composition of any one of Embodiments 1 to 11, wherein the one or more active ingredients of constituent (a) have a solubility of 10 g/L or less in deionized water having a pH of about 7 at 25° C. and 1013 mbar.

Embodiment 14. The composition of any one of Embodiments 1 to 11, wherein the one or more active ingredients of constituent (a) have a solubility of 5 g/L or less in deionized water having a pH of about 7 at 25° C. and 1013 mbar.

Embodiment 15. The composition of any one of Embodiments 1 to 11, wherein the one or more active ingredients of constituent (a) have a solubility of 2 g/L or less in deionized water having a pH of about 7 at 25° C. and 1013 mbar.

Embodiment 16. The composition of any one of Embodiments 1 to 11, wherein the one or more active ingredients of constituent (a) have a solubility of 1 g/L or less in deionized water having a pH of about 7 at 25° C. and 1013 mbar.

Embodiment 17. The composition of any one of Embodiments 1 to 16, wherein the total amount of water in the composition is less than about 15% by weight, based on the total weight of the composition.

Embodiment 18. The composition of any one of Embodiments 1 to 16, wherein the total amount of water in the composition is less than about 10% by weight, based on the total weight of the composition.

Embodiment 19. The composition of any one of Embodiments 1 to 16, wherein the total amount of water in the composition is less than about 5% by weight, based on the total weight of the composition.

Embodiment 20. The composition of any one of Embodiments 1 to 19, wherein the composition comprises water.

Embodiment 21. The composition of any one of Embodiments 1 to 20, wherein the one or more active ingredients of constituent (a) have a solubility in acetone of at least about 10 g/L at 25° C. and 1013 mbar.

Embodiment 22. The composition of any one of Embodiments 1 to 21, wherein the total amount of constituent (c) is at least about 5% by weight, based on the total weight of the composition.

Embodiment 23. The composition of any one of Embodiments 1 to 21, wherein the total amount of constituent (c) is at least about 10% by weight, based on the total weight of the composition.

Embodiment 24. The composition of any one of Embodiments 1 to 23, wherein constituents (a) and (b) are present substantially in the same phase.

Embodiment 25. The composition of any one of Embodiments 1 to 24, wherein the depression of the melting point of the composition is below that of pure urea by at least 2° C. as measured by differential scanning calorimetry.

Embodiment 26. The composition of any one of Embodiments 1 to 25, wherein composition is an inclusion complex.

Embodiment 27. The composition of any one of Embodiments 1 to 25, wherein composition is a solid solution.

Embodiment 28. The composition of any one of Embodiments 1 to 27, wherein nanoparticles of the one or more active ingredients of constituent (a) are formed upon adding the composition to water having a pH of about 7 at 25° C. and 1013 mbar in an amount of at least about 5 times the weight of the composition.

Embodiment 29. The composition of any one of Embodiments 1 to 28, wherein the one or more active ingredients of constituent (a) are substantially present in the form of a liquid upon adding the composition to water having a pH of about 7 at 25° C. and 1013 mbar in an amount of at least about 5 times the weight of the composition.

Embodiment 30. The composition of any one of Embodiments 1 to 28, wherein the one or more active ingredients of constituent (a) are substantially present in the form of a liquid upon adding the composition to a 1% by weight solution of one or more emulsifying surfactants in water at 25° C. and 1013 mbar in an amount of at least about 5 times the weight of the composition.

Embodiment 31. The composition of Embodiment 29 or 30, wherein the active ingredient(s) of constituent (a) are substantially present in the form of an (emulsified) liquid for at least 30 minutes.

Embodiment 32. The composition of any one of Embodiments 1 to 31, wherein at least one surfactant of constituent (c) has a hydrophilic-lipophilic-balance (HLB) value of about 3 or higher.

Embodiment 33. The composition of any one of Embodiments 1 to 31, wherein at least one surfactant of constituent (c) has a hydrophilic-lipophilic-balance (HLB) value of about 4 or higher.

Embodiment 34. The composition of any one of Embodiments 1 to 31, wherein at least one surfactant of constituent (c) has a hydrophilic-lipophilic-balance (HLB) value of about 5 or higher.

Embodiment 35. The composition of any one of Embodiments 1 to 31, wherein at least one surfactant of constituent (c) has a hydrophilic-lipophilic-balance (HLB) value of about 6 or higher.

Embodiment 36. The composition of any one of Embodiments 1 to 31, wherein constituent (c) has a hydrophilic-lipophilic-balance (HLB) of about 3 or higher.

Embodiment 37. The composition of any one of Embodiments 1 to 31, wherein constituent (c) has a hydrophilic-lipophilic-balance (HLB) of about 4 or higher.

Embodiment 38. The composition of any one of Embodiments 1 to 31, wherein constituent (c) has a hydrophilic-lipophilic-balance (HLB) of about 5 or higher.

Embodiment 39. The composition of any one of Embodiments 1 to 31, wherein constituent (c) has a hydrophilic-lipophilic-balance (HLB) of about 6 or higher.

Embodiment 40. The composition of any one of Embodiments 1 to 39, wherein constituent (c) comprises or consists of one or more non-ionic surfactants selected from the group consisting of (poly) alkoxylated alcohols, (poly) alkoxylated phosphate esters. and (poly) alkoxylated tristyrylphenols

Embodiment 41. The composition of any one of Embodiments 1 to 40, wherein constituent (c) comprises or consists of one or more non-ionic surfactants selected from the group consisting of (poly) alkoxylated linear saturated or monounsaturated C12-C18-alcohols and (poly) alkoxylated phosphate esters of linear saturated or monounsaturated C12-C18-alcohols.

Embodiment 42. The composition of any one of Embodiments 1 to 41, wherein constituent (c) comprises or consists of one or more non-ionic surfactants having a degree of alkoxylation in the range of from about 2 to about 14.

Embodiment 43. The composition of any one of Embodiments 1 to 41, wherein constituent (c) comprises or consists of one or more non-ionic surfactants having a degree of alkoxylation in the range of from about 4 to about 10.

Embodiment 44. The composition of any one of Embodiments 1 to 43, wherein constituent (c) comprises or consists of one or more non-ionic surfactants selected from the group consisting of (poly) ethoxylated linear saturated or monounsaturated C12-C18-alcohols and (poly) ethoxylated phosphate esters of linear saturated or monounsaturated C12-C18-alcohols.

Embodiment 45. The composition of any one of Embodiments 1 to 44, wherein constituent (c) comprises or consists of one or more non-ionic surfactants comprising from to about 2 to about 14 ethylene glycol units (PEG-2 to PEG-14).

Embodiment 46. The composition of any one of Embodiments 1 to 45, wherein constituent (c) comprises or consists of one or more non-ionic surfactants comprising from about 4 to about 10 ethylene glycol units (PEG-4 to PEG-10).

Embodiment 47. The composition of any one of Embodiments 1 to 46, wherein constituent (c) comprises or consists of one or more non-ionic surfactants selected from the group consisting of tristyrylphenol ethoxylates.

Embodiment 48. The composition of Embodiment 47, wherein constituent (c) comprises or consists of one or more non-ionic surfactants comprising from about 6 to about 80 ethylene glycol units (PEG-6 to PEG-80).

Embodiment 49. The composition of Embodiment 47, wherein constituent (c) comprises or consists of one or more non-ionic surfactants comprising from about 10 to about 60 ethylene glycol units (PEG-10 to PEG-60), preferably

Embodiment 50. The composition of any one of Embodiments 1 to 49, wherein the total amount of constituent (c) is in the range of from about 2% by weight to 20% by weight, based on the total weight of the composition.

Embodiment 51. The composition of any one of Embodiments 1 to 50, wherein constituent (c) comprises or consists of one or more non-ionic surfactants selected from the group consisting of ethoxylated linear saturated or monounsaturated C12-C18-alcohols with about 4 to about 10 ethylene glycol units (PEG-4 to PEG-10), and wherein the total amount of constituent (c) is in the range of from about 4% by weight to about 20% by weight, based on the total weight of the composition.

Embodiment 52. The composition of any one of Embodiments 1 to 51, wherein constituent (c) comprises or consists of one or more non-ionic surfactants selected from the group consisting of ethoxylated phosphate esters of linear saturated or monounsaturated C12-C18-alcohols with about 4 to about 10 ethylene glycol units (PEG-4 to PEG-10), and wherein the total amount of constituent (c) is in the range of from about 2% by weight to about 10% by weight, based on the total weight of the composition.

Embodiment 53. The composition of any one of Embodiments 1 to 52 further comprising as constituent (d) one or more polymeric dispersants different from constituent (c).

Embodiment 54. The composition of any one of Embodiments 1 to 53 further comprising as constituent (d) one or more polymeric dispersants different from constituent (c) selected from the group consisting of polycarboxylates and salts thereof (preferably sodium salts), maleic anhydride-isobutylene copolymers and salts thereof (preferably sodium salts), and block copolymers of styrene oxide and ethylene oxide, lignosulfates, and mixtures thereof.

Embodiment 55. The composition of any one of Embodiments 1 to 54, wherein the composition comprises as constituent (d) one or more polymeric dispersants different from constituent (c), wherein the total amount of constituent (d) is in the range of about 1 to about 15% by weight, based on the total weight of the composition.

Embodiment 56. The composition of any one of Embodiments 1 to 55, wherein the composition comprises as constituent (e) one or more wetting agents different from constituents (c) and (d), wherein the composition comprises as constituent (e) preferably one or more wetting agents with a non-linear structure, wherein constituent (e) is different from constituents (c) and (d).

Embodiment 57. The composition of any one of Embodiments 1 to 56, wherein the composition comprises as constituent (e) one or more wetting agents different from constituents (c) and (d) selected from the group consisting of Guerbet alcohol ethoxylates, alkyl polyglucosides, alkylnaphthalene sulfonate condensates, and mixtures thereof.

Embodiment 58. The composition of any one of Embodiments 1 to 56, wherein the composition comprises as constituent (e) one or more wetting agents different from constituents (c) and (d) and selected from the group consisting of ethoxylated branched alcohols.

Embodiment 59. The composition of any one of Embodiments 1 to 56, wherein the composition comprises as constituent (e) one or more wetting agents different from constituents (c) and (d) selected from the group consisting of alkylnaphthalene sulfonate condensates.

Embodiment 60. The composition of any one of Embodiments 1 to 59, wherein the composition comprises as constituent (e) one or more wetting agents different from constituents (c) and (d), wherein the total amount of constituent (e) is in the range of about 5 to about 30% by weight, based on the total weight of the composition.

Embodiment 61. The composition of any one of Embodiments 1 to 60, wherein the composition comprises as constituent (f) a water-soluble polymeric binder different from constituents (c), (d) and (e).

Embodiment 62. The composition of any one of Embodiments 1 to 61, wherein the composition comprises as constituent (f) a water-soluble polymeric binder different from constituents (c), (d) and (e), selected from the group consisting of cellulose ethers and salts thereof, preferably water-soluble methylcellulose polymers, water-soluble hydroxypropyl methylcellulose polymers, and mixtures thereof.

Embodiment 63. The composition of any one of Embodiments 1 to 62, wherein the composition comprises as constituent (f) a water-soluble polymeric binder different from constituents (c), (d) and (e) in a total amount in the range of from about 0.5 to about 3% by weight, based on the total weight of the composition.

Embodiment 64. The composition of any one of Embodiments 1 to 63, wherein the total amount of methanol in the composition is less than about 1% by weight, based on the total weight of the composition.

Embodiment 65. The composition of any one of Embodiments 1 to 64, wherein the total amount of ethanol in the composition is less than about 1% by weight, based on the total weight of the composition.

Embodiment 66. The composition of any one of Embodiments 1 to 63, wherein the total amount of C1-C6 alcohols in the composition is less than about 1% by weight, based on the total weight of the composition.

Embodiment 67. The composition of any one of Embodiments 1 to 66, wherein the total amount of oleic acid in the composition is less than about 1% by weight, based on the total weight of the composition.

Embodiment 68. The composition of any one of Embodiments 1 to 67, wherein the total amount of constituents in salt form is about 10% by weight or less, preferably about 5% by weight or less, in each case based on the total weight of the composition.

Embodiment 69. The composition of any one of Embodiments 1 to 68, wherein the composition comprises phosphoric acid or methanesulfonic acid.

Embodiment 70. The composition of Embodiment 69, wherein the total amount of phosphoric acid and methanesulfonic acid is in the range of from about 0.3% by weight to about 1.5% by weight, preferably from about 0.6% by weight to about 1.2% by weight, in each case based on the total weight of the composition.

Embodiment 71. The composition of any one of Embodiments 1 to 70, wherein constituent (a) comprises or consists of one or more pesticidal active ingredients.

Embodiment 72. The composition of any one of Embodiments 1 to 70, wherein constituent (a) comprises or consists of pesticidal active ingredients selected from the group of fungicides, herbicides, insecticides and safeners.

Embodiment 73. The composition of any one of Embodiments 1 to 70, wherein constituent (a) is selected from the group consisting of chlorotriazine herbicides, pyridine fungicides, aroylcyclohexanedione herbicides, carbanilate herbicides, conazole fungicides, pyridylpyrazole insecticides, and mixtures thereof.

Embodiment 74. The composition of any one of Embodiments 1 to 70, wherein constituent (a) comprises or consists of pesticidal active ingredients selected from the group consisting of atrazine, cyprosulfamide, fluopyram, isoxaflutole, mesotrione, phenmedipham, prothioconazole, tembotrione, tetraniliprole, and mixtures thereof.

Embodiment 75. The composition of any one of Embodiments 1 to 70, wherein constituent (a) comprises or consists of pesticidal active ingredients selected from the group consisting of atrazine, fluopyram, mesotrione, phenmedipham, prothioconazole, tembotrione, tetraniliprole, and mixtures thereof.

Embodiment 76. The composition of any one of Embodiments 1 to 70, wherein constituent (a) comprises or consists of tembotrione, mesotrione, phenmedipham, or combinations thereof.

Embodiment 77. The composition of any one of Embodiments 1 to 70, wherein constituent (a) comprises or consists of fluopyram.

Embodiment 78. The composition of any one of Embodiments 1 to 70, wherein constituent (a) comprises or consists of tetraniliprole.

Embodiment 79. A product obtainable or obtained by extrusion of a composition as defined in any one of Embodiments 1 to 78.

Embodiment 80. The product according to Embodiment 79, wherein the extrusion temperature is in the range of from about 85° C. to about 110° C., and subsequent cooling of the molten composition to a temperature below 55° C.

Embodiment 81. A product obtainable or obtained by melting a composition as defined in any one of Embodiments 1 to 78, and subsequent cooling of the molten composition to a temperature below 55° C.

Embodiment 82. The product according to Embodiment 81, wherein melting of the composition is performed such that the temperature of the molten composition is in the range of from about 75° C. to about 110° C.

Embodiment 83. The product according to any one of Embodiments 79 to 82, wherein the total amount of methanol and/or ethanol in the product is less than about 1% by weight, based on the total weight of the product.

Embodiment 84. The product according to any one of Embodiments 79 to 82, wherein the total amount of C1-C6 alcohols in the product is less than about 1% by weight, based on the total weight of the product.

Embodiment 85. The product according to any one of Embodiments 79 to 84, wherein the total amount of oleic acid in the product is less than about 1% by weight, based on the total weight of the product.

Embodiment 86. A process for preparing a composition as defined in any one of Embodiments 1 to 78, characterized by the following steps:
1. Combining constituents (a), (b), (c), and optionally one or more further constituents selected from the group consisting of water, and constituents (d), (e) and (f) as defined in Embodiments 1 to 78,
2. Mixing the combination resulting from step 1 at a temperature of in the range of from about 60° C. to about 140° C., preferably at a temperature of in the range of from about 75° C. to about 125° C., more preferably at a temperature of in the range of from about 80° C. to about 110° C.,
3. Cooling the composition resulting from step 2.

Embodiment 87. The process according to Embodiment 86, wherein step 3 includes extrusion, spray drying, spray chilling, prilling, spheronization, or combinations thereof.

Embodiment 88. A product obtainable or obtained by the process of Embodiment 86 or 87 wherein, upon adding the product at 25° C. to an amount of water sufficient to dissolve the urea, the one or more active ingredients of constituent (a) is substantially present as nanoparticles with diameters substantially below 100 nm, as determined by dynamic light scattering,

Embodiment 89. A product obtainable or obtained by the process of Embodiment 86 or 87 wherein, upon adding the product at 25° C. to an amount of an aqueous emulsifier dilution sufficient to dissolve the urea, an emulsion of the liquid one or more active ingredients of constituent (a) is obtained which is stable against crystallization of said one or more the active ingredients of constituent (a) for at least 30 minutes at 25° C.

Embodiment 90. An application mixture, comprising
a composition or product defined in any one of Embodiments 1 to 85, 88 or 89,
water in an amount by weight at least 10 times, preferably at least 25 times, more preferably at least 50 times, the amount of said composition or product,
and
one or more constituents selected from the group consisting of further adjuvants, other diluents, and other active ingredients.

Embodiment 91. The application mixture of Embodiment 90, wherein the application mixture is a spray application mixture.

Embodiment 92. A formulation comprising a composition or product defined in any one of Embodiments 1 to 85, 88 or 89, and one or more further constituents selected from the group consisting of adjuvants, liquid ingredients at 25° C. and 1013 mbar, solid active ingredients at 25° C. and 1013 mbar, and mixtures thereof.

Embodiment 93. The formulation of Embodiment 92, wherein the formulation is selected from the group consisting of wettable granules (WG), wettable powders (WP), and oil dispersions (OD).

Embodiment 94. The formulation of Embodiment 92 or 93, wherein the formulation is an oil dispersion (OD) formulation, and the one or more formulation adjuvants are selected from the group consisting of oily active ingredients, vegetable oils, oily solvents, and mixtures thereof.

Embodiment 95. The formulation of Embodiment 94, comprising an oily herbicide, wherein the oily herbicide comprises or is acetochlor.

Embodiment 96. The formulation of Embodiment 94 or 95, comprising an oily solvent, wherein the oily solvent comprises or is a long-chain fatty acid methyl ester, preferably methyl soyate.

Embodiment 97. The formulation of Embodiment 92 or 93, wherein the formulation is a wettable granule (WG) formulation or a wettable powder (WP) formulation, wherein the adjuvant comprises or is a dry emulsifier powder or a dry powder dispersant.

Embodiment 98. The composition, product, application mixture or formulation defined in any one of Embodiments 1 to 85, 88 to 91 or 92 to 97, comprising a pesticidal active ingredient, for use in a method of controlling undesired vegetation, plant pests, (phytopathogenic) fungi or (phytopathogenic) nematodes.

Embodiment 99. A method for controlling undesired vegetation, plant pests, phytopathogenic fungi or phytopathogenic nematodes comprising the application of a composition, product, application mixture or formulation defined in any one of Embodiments 1 to 85, 88 to 91 or 92 to 97, comprising a pesticidal active ingredient, to said undesired vegetation, plant pests, phytopathogenic fungi or phytopathogenic nematodes.

Embodiment 100. Use of the composition, product, application mixture or formulation defined in any one of Embodiments 1 to 85, 88 to 91 or 92 to 97, comprising a pesticidal active ingredient, to control undesired vegetation, plant pests, phytopathogenic fungi or phytopathogenic nematodes.

Embodiment 101. The composition, product or application mixture defined in any one of Embodiments 1 to 85 or 88 to 91, comprising a pharmaceutical active ingredient, for use as a medicament.

Embodiment 102. The composition, product or application mixture defined in any one of Embodiments 1 to 85 or 88 to 91, comprising a pharmaceutical active ingredient, for use in a method of treating an animal or human body.

Embodiment 103. A method of treating a subject in need thereof comprising administering a pharmaceutically effective amount of the composition, product or application mixture defined in any one of Embodiments 1 to 85 or 88 to 91, comprising a pharmaceutical active ingredient, to said subject.

Embodiment 104. Use of the composition, product or application mixture defined in any one of Embodiments 1 to 85 or 88 to 91, comprising a pharmaceutical active ingredient, in the treatment of an animal or human body, wherein said composition, product or application mixture preferably is orally administered to said animal or human body.

Example embodiments have been provided so that this disclosure will be thorough, and will fully convey the scope to those who are skilled in the art. Numerous specific details are set forth such as examples of specific components, assemblies, and methods, to provide a thorough understanding of embodiments of the present disclosure.

It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, seeds, members and/or sections, these elements, components, seeds, members and/or sections should not be limited by these terms. These terms may be only used to distinguish one element, component, seed, member or section from another element, component, seed, member or section. Terms such as “first,” “second,” and other numerical terms when used herein do not imply a sequence or order unless clearly indicated by the context. Thus, a first element, component, seed, member or section discussed below could be termed a second element, component, seed, member or section without departing from the teachings of the example embodiments.

In view of the above, it will be seen that the several objects of the invention are achieved, and other advantageous results attained.

As various changes could be made in the above products and methods without departing from the scope of the invention, it is intended that all matter contained in the above description shall be interpreted as illustrative and not in a limiting sense.

1. A composition in solid form at 25° C. and 1013 mbar comprising:
(a) a total amount of at least about 5% by weight of one or more pesticidal active ingredients having a melting point of at least 55° C. at 1013 mbar and a solubility of 50 g/L or less in deionized water having a pH of about 7 at 25° C. and 1013 mbar, constituent (a) not being present in form of a salt with an inorganic counter-ion;
(b) a total amount of at least about 50% by weight of urea; and
(c) a total amount of at least about 1% by weight of one or more non-ionic surfactants,
wherein a ratio by weight of a total amount of constituent (c) to a total amount of constituent (a) is about 0.8 or lower, and
wherein amounts indicated in each case are based on a total weight of the composition.

2. The composition of claim 1,
wherein constituent (a) is present in range of from 10% to 30% by weight,
constituent (b) is present in a range of from 60% to 85% by weight.

3. The composition of claim 1, wherein the ratio by weight of the total amount of constituent (c) to the total amount of constituent (a) is from about 0.1 to about 0.65, based on the total weight of the composition.

4. The composition of claim 3, wherein the ratio by weight of the total amount of constituent (c) to the total amount of constituent (a) is from about 0.2 to about 0.55, based on the total weight of the composition.

5. The composition of claim 1,
wherein the one or more active ingredients of constituent (a) have a solubility of 20 g/L or less in deionized water having a pH of about 7 at 25° C. and 1013 mbar,
and/or
wherein the one or more active ingredients of constituent (a) have a solubility in acetone of at least about 10 g/L at 25° C. and 1013 mbar.

6. The composition of claim 1, wherein constituents (a) and (b) are present in the same phase.

7. The composition of claim 1, wherein a depression of the melting point of the composition is below that of pure urea by at least 2° C. as measured by differential scanning calorimetry.

8. The composition of claim 1, wherein the composition is an inclusion complex or wherein the composition is a solid solution.

9. The composition of claim 1, wherein nanoparticles of the one or more active ingredients of constituent (a) are formed upon adding the composition to water having a pH of about 7 at 25° C. and 1013 mbar in an amount of at least about 5 times the weight of the composition.

10. The composition of claim 1, wherein the one or more active ingredients of constituent (a) are substantially present in the form of a liquid upon adding the composition to water having a pH of about 7 at 25° C. and 1013 mbar in an amount of at least about 5 times the weight of the composition
or
wherein the one or more active ingredients of constituent (a) are substantially present in the form of a liquid upon adding the composition to a 1% by weight solution of one or more emulsifying surfactants in water at 25° C. and 1013 mbar in an amount of at least about 5 times the weight of the composition.

11. The composition of claim 1, wherein at least one surfactant of constituent (c) has a hydrophilic-lipophilic-balance (HLB) value of about 3 or higher.

12. The composition of claim 1, wherein constituent (c) comprises one or more non-ionic surfactants selected from the group consisting of (poly) alkoxylated alcohols, (poly) alkoxylated phosphate esters, and (poly) alkoxylated tristyrylphenols.

13. The composition of claim 1, further comprising as constituent (d) one or more polymeric dispersants different from constituent (c).

14. The composition of claim 1, wherein the composition comprises as constituent (e) one or more wetting agents different from constituents (c) and (d).

15. The composition of claim 1, wherein the composition comprises as constituent (f) a water-soluble polymeric binder different from constituents (c), (d) and (e).

16. (canceled)

17. (canceled)

18. A process for preparing the composition as defined in claim 1, the process comprising:
i. Combining constituents (a), (b), and (c), to form a combination,
ii. Mixing the combination resulting from step 1 at a temperature of in a range of from about 60° C. to about 140° C.,
iii. Cooling the composition resulting from step 2.

19. (canceled)

20. (canceled)

21. An application mixture, comprising:
the composition defined in claim 1;
water in an amount by weight at least 10 times an amount of said composition or product; and
one or more constituents selected from the group consisting of further adjuvants, other diluents, and at least one other active ingredient.

22. (canceled)

23. (canceled)

24. A method for controlling undesired vegetation, plant pests,
phytopathogenic fungi or phytopathogenic nematodes, the method comprising;
application of the composition of claim 1 to said undesired vegetation, plant pests, phytopathogenic fungi or phytopathogenic nematodes.

25-29. (canceled)

30. The composition of claim 1 wherein the one or more active ingredients of constituent (a) preferably have a molecular weight of less than 800 Dalton.

31. The composition of claim 1, wherein constituent (c) is present in a total amount of 8% or more by weight.
